(19) United States
(12) Patent Application Publication
(10) Pub. No.: US 2002/0041870 A1
(43) Pub. Date: Apr. 11, 2002

(54) Novel genes expressed in obese rat hypothalamus

(76) Inventor: Linda H. Wu, Woodbridge, CT (US)

Correspondence Address: BANNER & WITCOFF, 1001 G STREET N W, SUITE 1100, WASHINGTON, DC, 20001, US

(21) Appl. No.: 09/912,447

(22) Filed: Jul. 26, 2001

Related U.S. Application Data
(60) Provisional application No. 60/220,878, filed on Jul. 26, 2000.

(51) Int. Cl. A61K 38/48; C07H 21/04; C12N 9/00; C12P 21/02; C12N 5/06

(52) U.S. Cl. 424/94.63; 435/183; 435/325; 435/320.1; 536/23.2

(57) ABSTRACT

Novel genes expressed in obese rat hypothalamus can be used to provide therapeutic reagents for treating obesity and related disorders.

This application claims the benefit of and incorporates by reference co-pending provisional application Ser. No. 60/220,878 filed Jul. 26, 2000.

### TECHNICAL FIELD OF THE INVENTION

The invention relates to methods and compositions for the modulation of processes related to mammalian body weight regulation, including treatment of body weight disorders such as obesity and cachexia, and modulation of thermogenesis.

### BACKGROUND OF THE INVENTION

The regulation of body fat in mammals is a complex process involving the regulation of not only appetite but also energy expenditure. See U.S. Pat. No. 6,057,109. An important component of energy expenditure is non-shivering thermogenesis (NST). In rodents, the majority of NST appears to occur in brown adipose tissue (BAT) via the uncoupling protein (UCP) (Cannon & Nedergaard, Essays in Biochem. 20, 110-65, 1985; Himms-Hagen, Prog. Lipid Res. 28, 67-115, 1989). UCP is a proton channel located exclusively in the inner mitochondrial membrane of adipocytes of the BAT (Nicholls & Locke, Physiol. Rev. 64, 1-64, 1984). By allowing protons to equilibrate across the inner mitochondrial membrane, UCP uncouples oxidative phosphorylation from ATP production and thus converts stored energy into heat rather than work (Klingenberg, Trends Biochem. Sci. 15, 108-12, 1990; Klaus et al., Int. J. Biochem. 23, 791-801, 1991). UCP-mediated uncoupling is not only capable of increasing body temperature in cold-acclimatized rodents and hibernating animals, but can also dissipate surplus caloric energy (Rothwell & Stock, In BROWN ADIPOSE TISSUE, Trayhurn et al., eds., London, Arnold, p. 269-298, 1986; Spiegelman & Flier, Cell 87, 377-89, 1996; Hamann & Flier, Endocrinology 137:2129, 1996). A number of studies have now implicated UCP and brown adipose tissue as important regulators of body weight in rodents (Hamann & Flier, Endocrinology 137, 2129, 1996; Lowell et al., Nature 366, 740-42, 1993; Kopecky et al., J. Clin. Invest. 96, 2914-23, 1995; Cunmnings et al., Nature 382, 622-26, 1996).

In humans, body weight homeostasis is poorly understood, but is also thought to involve regulated thermogenesis (Rothwell & Stock, Ann. Rev. Nutr. 1, 235-56, 1981; Segal et al., J. Clin. Invest. 89, 824-33, 1992; Jensen et al., Am. J. Physiol. 268, E433-38, 1995). However, the importance of the UCP in adult humans is questionable due to the low levels of BAT and consequently the low levels of UCP expression (Huttunen et al., Eur. J. Appl. Physiol. 46, 339-45, 1981; Cunningham et al., Clin. Sci. 69, 343-48, 1985; Schulz, J. Am. Diet Assoc. 87, 761-64, 1987; Santos et al., Arch. Pathol. Lab Med. 116, 1152-54, 1992).

In adult humans and other animals that do not contain large amounts of BAT, a large portion of NST and regulated thermogenesis is thought to be mediated by muscle and the white adipose tissue (Jensen et al., 1995; Davis, Am. J. Physiol. 213, 1423-26, 1963; Astrup et al., Am. J. Physiol. 257, E340-45, 1989; Simonsen et al., Am. J. Physiol. 263, E850-55, 1992; Simonsen et al., Int. J. Obes. Relat. Metab. Disord. 17 (Suppl. 3), S47-51, 1993; Duchamp et al., Am. J. Physiol. 265, R1076-83, 1993), however, the molecular mediators for regulated thermogenesis are currently unknown (Block, Ann. Rev. Physiol. 56, 535-77, 1994).

Further, body weight disorders, including eating and other disorders affecting regulation of body fat, represent major health problems in all industrialized countries. Obesity, the most prevalent of eating disorders, for example, is the most important nutritional disorder in the western world, with estimates of its prevalence ranging from 30% to 50% within the middle-aged population. Other body weight disorders, such as anorexia nervosa and bulimia nervosa which together affect approximately 0.2% of the female population of the western world, also pose serious health threats. Further, such disorders as anorexia and cachexia (wasting) are also prominent features of other diseases such as cancer, cystic fibrosis, and AIDS.

Obesity, defined as an excess of body fat relative to lean body mass, also contributes to other diseases. For example, this disorder is responsible for increased incidences of diseases such as coronary artery disease, stroke, and diabetes. Obesity is not merely a behavioral problem, i.e., the result of voluntary hyperphagia. Rather, the differential body composition observed between obese and normal subjects results from differences in both metabolism and neurologic/metabolic interactions. These differences seem to be, to some extent, due to differences in gene expression, and/or level of gene products or activity. The nature, however, of the genetic factors which control body composition are unknown, and attempts to identify molecules involved in such control have generally been empiric and the parameters of body composition and/or substrate flux are monitored have not yet been identified (Friedman et al., Mammalian Gene 1, 130-44, 1991).

The epidemiology of obesity strongly shows that the disorder exhibits inherited characteristics, (Stunkard, N. Eng. J. Med. 322, 1483, 1990). Moll et al., have reported that, in many populations, obesity seems to be controlled by a few genetic loci (Moll et al. Am. J. Hum. Gen. 49, 1243, 1991). In addition, human twin studies strongly suggest a substantial genetic basis in the control of body weight, with estimates of heritability of 80-90% (Simopoulos & Childs, eds., in “Genetic Variation and Nutrition in Obesity,” World Review of Nutrition and Diabetes 63, S. Karger, Basel, Switzerland, 1989; Boijeson, Acta. Paediatr. Scand. 65, 279-87, 1976).

Further, studies of non-obese persons who deliberately attempted to gain weight by systematically over-eating were found to be more resistant to such weight gain and able to maintain an elevated weight only by very high caloric intake. In contrast, spontaneously obese individuals are able to maintain their status with normal or only moderately elevated caloric intake.

In addition, it is a commonplace experience in animal husbandry that different strains of swine, cattle, etc., have different predispositions to obesity. Studies of the genetics of human obesity and of models of animal obesity demonstrate that obesity results from complex defective regulation of both food intake, food induced energy expenditure and of the balance between lipid and lean body anabolism.

There are a number of genetic diseases in man and other species that feature obesity among their more prominent symptoms, along with, frequently, dysmorphic features and mental retardation. Although no mammalian gene associated with an obesity syndrome has yet been characterized in molecular terms, a number of such diseases exist in humans. For example, Prader-Willi syndrome (PWS) affects approximately 1 in 20,000 live births, and involves poor neonatal muscle tone, facial and genital deformities, and generally obesity. The genetics of PWS are very complex, involving, for example, genetic imprinting, in which development of the disease seems to depend upon which parent contributes the abnormal PWS allele. In approximately half of all PWS patients, however, a deletion on the long arm of chromosome 11 is visible, making the imprinting aspect of the disease difficult to reconcile. Given the various symptoms generated, it seems likely that the PWS gene product may be required for normal brain function, and may, therefore, not be directly involved in adipose tissue metabolism.

In addition to PWS, many other pleiotropic syndromes that include obesity as a symptom have been characterized. These syndromes are more genetically straightforward, and appear to involve autosomal recessive alleles. The diseases include, among others, Ahlstroem, Carpenter, Bardet-Biedl, Cohen, and Morgagni-Stewart-Monel Syndromes.

Animals having mutations that lead to syndromes that include obesity symptoms have also been identified. Attempts have been made to utilize such animals as models for the study of obesity. The best-studied animal models for genetic obesity are mice which contain the autosomal recessive mutations ob/ob (obese) and db/db (diabetes). These mutations are on chromosomes 6 and 4, respectively, but lead to clinically similar pictures of obesity, evident starting at about 1 month of age, which include hyperphagia, severe abnormalities in glucose and insulin metabolism, very poor thermo-regulation and non-shivering thermogenesis, and extreme torpor and underdevelopment of the lean body mass. Restriction of the diet of these animals to restore a more normal body fat mass to lean body mass ration is fatal and does not result in a normal habitus.

Although the phenotypes of db/db and ob/ob mice are similar, the lesions are distinguishable by means of parabiosis. The feeding of normal mice and, putatively, all mammals, is regulated by satiety factors. The ob/ob mice are apparently unable to express the satiety factor, while the db/db mouse is unresponsive to it.

In addition to ob and db, several other single gene mutations resulting in obesity in mice have been identified. These include the yellow mutation at the agouti locus, which causes a pleiotropic syndrome that causes moderate adult onset obesity, a yellow coat color, and a high incidence of tumor formation (Herberg and Coleman, Metabolism 26, 59, 1977), and an abnormal anatomic distribution of body fat (Coleman, Diabetologia 14, 141-48, 1978). Additionally, mutations at the fat and tubby loci cause moderately severe, maturity-onset obesity with somewhat milder abnormalities in glucose homeostasis than are observed in ob and db mice (Coleman and Eicher, J. Heredity 81, 424-27, 1990). Further, autosomal dominant mutations at the adipose locus of chromosome 7, have been shown to cause obesity.

Other animal models include fa/fa (fatty) rats, which bear many similarities to the ob/ob and db/db mice, discussed above. One difference is that, while fa/fa rats are very sensitive to cold, their capacity for non-shivering thermogenesis is normal. Torpor seems to play a larger part in the maintenance of obesity in fa/fa rats than in the mice mutants. In addition, inbred mouse strains such as NZO mice and Japanese KK mice are moderately obese.

Certain hybrid mice, such as the Wellesley mouse, become spontaneously fat. Further, several desert rodents, such as the spiny mouse, do not become obese in their natural habitats, but do become so when fed on standard laboratory feed.

Animals that have been used as models for obesity have also been developed via physical or pharmacological methods. For example, bilateral lesions in the ventromedial hypothalamus (VMH) and ventrolateral hypothalamus (VLH) in the rat are associated, respectively, with hyperphagia and gross obesity and with aphagia and cachexia. Further, it has been demonstrated that feeding monosodium-glutamate (MSG) to new born mice also results in an obesity syndrome.

Attempts have been made to utilize such animal models in the study molecular causes of obesity. For example, adipsin, a murine serine protease with activity closely similar to human complement factor D, produced by adipocytes, has been found to be suppressed in ob/ob, db/db and MSG-induced obesity (Flier, Science 237, 405, 1987). The suppression of adipsin precedes the onset of obesity in each model (Lowell, Endocrinology 126, 1514), 1990. Further studies have mapped the locus of the defect in these models to activity of the adipsin promoter (Platt, Proc. Natl. Acad. Sci. U.S.A. 86, 7490, 1989). Further, alterations have been found in the expression of neurotransmitter peptides in the hypothalamus of the ob/ob mouse (Wilding, Endocrinology 132, 1939, 1993), of glucose transporter proteins in islet β-cells (Ohneda, Diabetes 42, 1065, 1993) and of the levels of G-proteins (McFarlane-Anderson, Biochem. J. 282, 15, 1992).

To date, no gene, in humans, has been found which is causative in the processes leading to obesity. Likewise, to date, no molecular mediator of regulated thermogenesis in humans has been identified. Given the importance of understanding body weight homeostasis and, further, given the severity and prevalence of disorders, including obesity, which affect body weight and body composition, there exists a great need for the systematic identification of genes involved in these processes and disorders.

### SUMMARY OF THE INVENTION

It is an object of the invention to provide reagents and methods of regulating the activities of obesity-specific polypeptides and genes. This and other objects of the invention are provided by one or more of the embodiments described below.

Another embodiment of the invention is an isolated polynucleotide encoding a polypeptide that comprises an amino acid sequence encoded by a nucleotide sequence selected from the group consisting of SEQ ID NOS:1-21 and the complements thereof.

Yet another embodiment of the invention is an expression vector comprising a polynucleotide encoding a polypeptide comprising an amino acid sequence encoded by a nucleotide sequence selected from the group consisting of SEQ ID NOS:1-21 and the complements thereof.

Another embodiment of the invention is a host cell comprising an expression vector that encodes a polypeptide comprising an amino acid sequence encoded by a nucleotide sequence selected from the group consisting of SEQ ID NOS:1-21 and the complements thereof.

Even another embodiment of the invention is a purified polypeptide comprising an amino acid sequence encoded by a nucleotide sequence selected from the group consisting of SEQ ID NOS:1-21 and the complements thereof.

A further embodiment of the invention is a purified polypeptide comprising a first amino acid sequence that comprises at least one conservative amino acid substitution compared with a second amino acid sequence encoded by a nucleotide sequence selected from the group consisting of SEQ ID NOS:1-21 and the complements thereof. Expression of the polypeptide is increased in a hypothalamus of an obese rat relative to expression of the polypeptide in a hypothalamus of a non-obese rat.

Still another embodiment of the invention is a fusion protein comprising a polypeptide consisting of an amino acid sequence encoded by a nucleotide sequence selected from the group consisting of SEQ ID NOS:1-21 and the complements thereof.

A further embodiment of the invention is a protein comprising a polypeptide comprising a first amino acid sequence that comprises at least one conservative amino acid substitution compared with a second amino acid sequence encoded by a nucleotide sequence selected from the group consisting of SEQ ID NOS:1-21 and the complements thereof, wherein expression of the polypeptide is increased in a hypothalamus of an obese rat relative to expression of the polypeptide in a hypothalamus of a non-obese rat.

Another embodiment of the invention is a method of producing a polypeptide comprising an amino acid sequence encoded by a nucleotide sequence selected from the group consisting of SEQ ID NOS:1-21 and the complements thereof. A host cell comprising an expression vector that encodes the polypeptide is cultured under conditions whereby the polypeptide is expressed. The polypeptide is isolated.

Yet another embodiment of the invention is a method of detecting a coding sequence for a polypeptide comprising an amino acid sequence encoded by a nucleotide sequence selected from the group consisting of SEQ ID NOS:1-21 and the complements thereof. A polynucleotide is hybridized to nucleic acid material of a biological sample, thereby forming a hybridization complex. The polynucleotide comprises 11 contiguous nucleotides of a nucleotide sequence selected from the group consisting of SEQ ID NOS:1-21 and the complements thereof. The hybridization complex is detected.

Another embodiment of the invention is a kit for detecting a coding sequence for a polypeptide comprising an amino acid sequence encoded by a nucleotide sequence selected from the group consisting of SEQ ID NOS:1-21 and the complements thereof. The kit comprises a polynucleotide comprising 11 contiguous nucleotides of a nucleotide sequence selected from the group consisting of SEQ ID NOS:1-21 and the complements thereof and instructions for detecting the coding sequence.

Yet another embodiment of the invention is a method of detecting a polypeptide comprising an amino acid sequence encoded by a nucleotide sequence selected from the group consisting of SEQ ID NOS:1-21 and the complements thereof. A biological sample is contacted with a reagent that specifically binds to the polypeptide to form a reagent-polypeptide complex. The reagent-polypeptide complex is detected.

Still another embodiment of the invention is a kit for detecting a polypeptide comprising an amino acid sequence encoded by a nucleotide sequence selected from the group consisting of SEQ ID NOS:1-21 and the complements thereof. The kit comprises an antibody which specifically binds to the polypeptide; and instructions for detecting the polypeptide.

Even another embodiment of the invention is a method of screening for agents that can regulate the activity of an obesity-specific polypeptide. A test compound is contacted with a first polypeptide selected from the group consisting of: (1) a second polypeptide encoded by a nucleotide sequence selected from the group consisting of SEQ ID NOS:1-21 and the complements thereof, and (2) a third polypeptide comprising at least one conservative amino acid substitution compared with the second polypeptide. Expression of the third polypeptide is increased in a hypothalamus of an obese rat relative to expression of the polypeptide in a hypothalamus of a non-obese rat. Binding of the test compound to the first polypeptide is detected. A test compound that binds to the first polypeptide is thereby identified as a potential agent for regulating activity of the obesity-specific polypeptide.

Yet another embodiment of the invention is a method of screening for agents that regulate an activity of an obesity-specific polypeptide. A test compound is contacted with a product encoded by a nucleotide sequence selected from the group consisting of SEQ ID NOS:1-21 and the complements thereof. Binding of the test compound to the product is detected. A test compound that binds to the product is thereby identified as a potential agent for regulating the activity of the obesity-specific polypeptide.

Another embodiment of the invention is a method of reducing expression of an obesity-specific polypeptide. A cell is contacted with a reagent that specifically binds to a product encoded by a nucleotide sequence selected from the group consisting of SEQ ID NOS:1-21 and the complements thereof. Expression of the obesity-specific polypeptide is thereby reduced.

Still another embodiment of the invention is a pharmaceutical composition comprising an antibody that specifically binds to a polypeptide encoded by a nucleotide sequence selected from the group consisting of SEQ ID NOS:1-21 and the complements thereof and a pharmaceutically acceptable carrier.

Another embodiment of the invention is a pharmaceutical composition comprising an antisense oligonucleotide that specifically binds to a nucleotide sequence selected from the group consisting of SEQ ID NOS:1-21 and the complements thereof and a pharmaceutically acceptable carrier.

A further embodiment of the invention is a pharmaceutical composition comprising an expression vector encoding a polypeptide comprising an amino acid sequence encoded by a nucleotide sequence selected from the group consisting of SEQ ID NOS:1-21 and the complements thereof and a pharmaceutically acceptable carrier.

Still another embodiment of the invention is an antibody that specifically binds to a polypeptide encoded by a nucleotide sequence selected from the group consisting of SEQ ID NOS:1-21 and the complements thereof.

The invention thus provides obesity-specific polypeptides and polynucleotides that can be used to identify test compounds that may act, for example, as activators or inhibitors of the polypeptides. Obesity-specific polypeptides and fragments thereof also are useful in raising specific antibodies, which can effectively reduce the levels of obesity-specific polypeptides.

### DETAILED DESCRIPTION OF THE INVENTION

Novel obesity-specific genes and polypeptides are a discovery of the present invention. Partial sequences of these genes were isolated from an obese rat hypothalamus cDNA library. These partial sequences have the nucleotide sequences shown in SEQ ID NOS:1-21 or the complements thereof.

Polypeptides

Obesity-specific polypeptides according to the invention comprise at least 6, 10, 15, 20, 25, 50, 75, or 100 contiguous amino acids selected from an amino acid sequence encoded by a polynucleotide comprising a nucleotide sequence shown in SEQ ID NOS:1-21 or a complement thereof or a naturally occurring variant of one of those amino acid sequences, as defined below. An obesity-specific polypeptide of the invention therefore can be a portion of an obesity-specific protein, a full-length obesity-specific protein, or a fusion protein comprising all or a portion of an obesity-specific protein.

Naturally Occurring Variants

Obesity-specific polypeptide variants that occur naturally and have the same activity as the obesity-specific polypeptides disclosed herein also are obesity-specific polypeptides. Preferably, naturally occurring obesity-specific polypeptide variants have amino acid sequences which are at least about 50, 55, 60, 65, or 70, preferably about 75, 80, 85, 90, 96, 96, or 98% identical to the amino acid sequences encoded by a polynucleotide comprising a nucleotide sequence shown in SEQ ID NOS:1-21 or the complement thereof. Percent identity between a putative obesity-specific polypeptide variant and an amino acid sequence encoded by a polynucleotide comprising a nucleotide sequence of SEQ ID NOS:1-21 or the complement thereof is determined using the Blast2 alignment program (Blosum62, Expect 10, standard genetic codes).

Variations in percent identity can be due, for example, to amino acid substitutions, insertions, or deletions. Amino acid substitutions are defined as one for one amino acid replacements. They are conservative in nature when the substituted amino acid has similar structural and/or chemical properties. Examples of conservative replacements are substitution of a leucine with an isoleucine or valine, an aspartate with a glutamate, or a threonine with a serine.

Amino acid insertions or deletions are changes to or within an amino acid sequence. They typically fall in the range of about 1 to 5 amino acids. Guidance in determining which amino acid residues can be substituted, inserted, or deleted without abolishing biological or immunological activity of an obesity-specific polypeptide can be found using computer programs well known in the art, such as DNASTAR software.

Fusion Proteins

Fusion proteins are useful for generating antibodies against obesity-specific polypeptide amino acid sequences and for use in various assay systems. For example, fusion proteins can be used to identify proteins that interact with portions of an obesity-specific polypeptide. Protein affinity chromatography or library-based assays for protein-protein interactions, such as the yeast two-hybrid or phage display systems, can be used for this purpose. Such methods are well known in the art and also can be used as drug screens.

An obesity-specific polypeptide fusion protein comprises two polypeptide segments fused together by means of a peptide bond. The first polypeptide segment comprises at least 6, 10, 15, 20, 25, 50, 75, or 100 contiguous amino acids encoded by a polynucleotide comprising a nucleotide sequence shown in SEQ ID NO:1-21 or its complement or of a naturally occurring variant, such as those described above. The first polypeptide segment also can comprise full-length obesity-specific polypeptide.

The second polypeptide segment can be a full-length protein or a protein fragment. Proteins commonly used in fusion protein construction include β-galactosidase, β-glucuronidase, green fluorescent protein (GFP), autofluorescent proteins, including blue fluorescent protein (BFP), glutathione-S-transferase (GST), luciferase, horseradish peroxidase (HRP), and chloramphenicol acetyltransferase (CAT). Additionally, epitope tags are used in fusion protein constructions, including histidine (His) tags, FLAG tags, influenza hemagglutinin (HA) tags, Myc tags, VSV-G tags, and thioredoxin (Trx) tags. Other fusion constructions can include maltose binding protein (MBP), S-tag, Lex a DNA binding domain (DBD) fusions, GAL4 DNA binding domain fusions, and herpes simplex virus (HSV) BP16 protein fusions. A fusion protein also can be engineered to contain a cleavage site located between the obesity-specific polypeptide-encoding sequence and the heterologous protein sequence, so that the obesity-specific polypeptide can be cleaved and purified away from the heterologous moiety.

A fusion protein can be synthesized chemically, as is known in the art. Preferably, a fusion protein is produced by covalently linking two polypeptide segments or by standard procedures in the art of molecular biology. Recombinant DNA methods can be used to prepare fusion proteins, for example, by making a DNA construct which comprises coding sequences selected from a polynucleotide comprising a nucleotide sequence shown in SEQ ID NO:1-21 or its complement in proper reading frame with nucleotides encoding the second polypeptide segment and expressing the DNA construct in a host cell, as is known in the art. Many kits for constructing fusion proteins are available from companies such as Promega Corporation (Madison, Wis.), Stratagene (La Jolla, Calif.), CLONTECH (Mountain View, Calif.), Santa Cruz Biotechnology (Santa Cruz, Calif.), MBL International Corporation (MIC; Watertown, Mass.), and Quantum Biotechnologies (Montreal, Canada; 1-888-DNA-KITS).

Identification of Species Homologs

Species homologs of the obesity-specific polypeptides disclosed herein, including human homologs, can be obtained using obesity-specific polynucleotides (described below) to make suitable probes or primers for screening cDNA expression libraries from other species, such as mice, monkeys, or yeast, identifying cDNAs which encode homologs of obesity-specific polypeptides, and expressing the cDNAs as is known in the art.

Polynucleotides

An obesity-specific polynucleotide can be single- or double-stranded and comprises a coding sequence or the complement of a coding sequence for an obesity-specific polypeptide. Partial sequences of obesity-specific polynucleotides are shown in SEQ ID NOS:1-21.

Degenerate nucleotide sequences encoding obesity-specific polypeptides, as well as homologous nucleotide sequences which are at least about 50, 55, 60, 65, 70, preferably about 75, 90, 96, or 98% identical to the nucleotide sequences shown in SEQ ID NOS:1-21 or their complements also are obesity-specific polynucleotides. Percent sequence identity between the sequences of two polynucleotides is determined using computer programs such as ALIGN which employ the FASTA algorithm, using an affine gap search with a gap open penalty of −12 and a gap extension penalty of −2. Complementary DNA (cDNA) molecules, species homologs, and variants of obesity-specific polynucleotides that encode biologically active obesity-specific polypeptides also are obesity-specific polynucleotides.

Identification of Polynucleotide Variants and Homologs

Variants and homologs of the obesity-specific polynucleotides described above also are obesity-specific polynucleotides. Typically, homologous polynucleotide sequences can be identified by hybridization of candidate polynucleotides to known polynucleotides under stringent conditions, as is known in the art. For example, using the following wash conditions—2×SSC (0.3 M NaCl, 0.03 M sodium citrate, pH 7.0), 0.1% SDS, room temperature twice, 30 minutes each; then 2×SSC, 0.1% SDS, 50° C. once, 30 minutes; then 2×SSC, room temperature twice, 10 minutes each—homologous sequences can be identified which contain at most about 25-30% basepair mismatches. More preferably, homologous nucleic acid strands contain 15-25% basepair mismatches, even more preferably 5-15% basepair mismatches.

Species homologs of the obesity-specific polynucleotides disclosed herein also can be identified by making suitable probes or primers and screening cDNA expression libraries from other species, such as mice, humans, monkeys, or yeast. Human variants of obesity-specific polynucleotides of the invention can be identified, for example, by screening human cDNA expression libraries. It is well known that the Tₘ of a double-stranded DNA decreases by 1-1.5° C. with every 1% decrease in homology (Bonner et al., J. Mol. Biol. 81, 123 (1973). Variants of the obesity-specific polynucleotides disclosed herein or obesity-specific polynucleotides of other species can therefore be identified by hybridizing a putative homologous polynucleotide with a polynucleotide comprising a nucleotide sequence of SEQ ID NO:1-21 or the complement thereof to form a test hybrid. The melting temperature of the test hybrid is compared with the melting temperature of a hybrid comprising polynucleotides having perfectly complementary nucleotide sequences, and the number or percent of basepair mismatches within the test hybrid is calculated.

Nucleotide sequences which hybridize to obesity-specific polynucleotides or their complements following stringent hybridization and/or wash conditions also are obesity-specific polynucleotides of the invention. Stringent wash conditions are well known and understood in the art and are disclosed, for example, in Sambrook et al., MOLECULAR CLONING: A LABORATORY MANUAL, 2d ed., 1989, at pages 9.50-9.51.

Typically, for stringent hybridization conditions a combination of temperature and salt concentration should be chosen that is approximately 12-20° C. below the calculated Tₘ of the hybrid under study. The Tₘ of a hybrid between an obesity-specific polynucleotide having a nucleotide sequence shown in SEQ ID NO:1-21 or the complement thereof and a polynucleotide sequence which is at least about 50, 55, 60, 65, 70, preferably about 75, 90, 96, or 98% identical to one of those nucleotide sequences can be calculated, for example, using the equation of Bolton and McCarthy, Proc. Natl. Acad. Sci. U.S.A. 48, 1390 (1962):

Tₘ=81.5° C.−16.6(log₁₀[Na⁺])+0.41(% G +C)−0.63(% formamide)−600/l),

where l=the length of the hybrid in basepairs. Stringent wash conditions include, for example, 4×SSC at 65° C., or 50% formamide, 4×SSC at 42° C., or 0.5×SSC, 0.1% SDS at 65° C. Highly stringent wash conditions include, for example, 0.2×SSC at 65° C.

Preparation of Polynucleotides

An obesity-specific polynucleotide can be isolated free of other cellular components such as membrane components, proteins, and lipids. Polynucleotides can be made by a cell and isolated using standard nucleic acid purification techniques, or synthesized using an amplification technique, such as the polymerase chain reaction (PCR), or by using an automatic synthesizer. Methods for isolating polynucleotides are routine and are known in the art. Any such technique for obtaining a polynucleotide can be used to obtain isolated obesity-specific polynucleotides. For example, restriction enzymes and probes can be used to isolate polynucleotide fragments that comprise obesity-specific nucleotide sequences. Isolated polynucleotides are in preparations that are free or at least 70, 80, or 90% free of other molecules.

Obesity-specific cDNA molecules can be made with standard molecular biology techniques, using obesity-specific mRNA as a template. cDNA molecules can thereafter be replicated using molecular biology techniques known in the art and disclosed in manuals such as Sambrook et al. (1989). An amplification technique, such as PCR, can be used to obtain additional copies of polynucleotides of the invention, using either genomic DNA or cDNA as a template.

Alternatively, synthetic chemistry techniques can be used to synthesize obesity-specific polynucleotides. The degeneracy of the genetic code allows alternate nucleotide sequences to be synthesized which will encode an obesity-specific polypeptide comprising, for example, an amino acid sequence encoded by a polynucleotide molecule comprising nucleotide sequence shown in SEQ ID NO:1-21 or the complement thereof or comprising an amino acid sequence that is a variant thereof.

Extending Polynucleotides

The partial sequences disclosed herein can be used to identify the corresponding full-length gene from which they were derived. The partial sequences can be nick-translated or end-labeled with ³²P using polynucleotide kinase using labeling methods known to those with skill in the art (BASIC METHODS IN MOLECULAR BIOLOGY, Davis et al., eds., Elsevier Press, N.Y., 1986). A lambda library prepared from human tissue can be directly screened with the labeled sequences of interest or the library can be converted en masse to pBluescript (Stratagene Cloning Systems, La Jolla, Calif. 92037) to facilitate bacterial colony screening (see Sambrook et al., MOLECULAR CLONING: A LABORATORY MANUAL, Cold Spring Harbor Laboratory Press (1989, pg. 1.20).

Both methods are well known in the art. Briefly, filters with bacterial colonies containing the library in pBluescript or bacterial lawns containing lambda plaques are denatured, and the DNA is fixed to the filters. The filters are hybridized with the labeled probe using hybridization conditions described by Davis et al., 1986. The partial sequences, cloned into lambda or pBluescript, can be used as positive controls to assess background binding and to adjust the hybridization and washing stringencies necessary for accurate clone identification. The resulting autoradiograms are compared to duplicate plates of colonies or plaques; each exposed spot corresponds to a positive colony or plaque. The colonies or plaques are selected, expanded and the DNA is isolated from the colonies for further analysis and sequencing.

Positive cDNA clones are analyzed to determine the amount of additional sequence they contain using PCR with one primer from the partial sequence and the other primer from the vector. Clones with a larger vector-insert PCR product than the original partial sequence are analyzed by restriction digestion and DNA sequencing to determine whether they contain an insert of the same size or similar as the mRNA size determined from Northern blot Analysis.

Once one or more overlapping cDNA clones are identified, the complete sequence of the clones can be determined, for example after exonuclease III digestion (McCombie et al., Methods 3, 33-40, 1991). A series of deletion clones are generated, each of which is sequenced. The resulting overlapping sequences are assembled into a single contiguous sequence of high redundancy (usually three to five overlapping sequences at each nucleotide position), resulting in a highly accurate final sequence.

Various PCR-based methods can be used to extend the nucleic acid sequences disclosed herein to detect upstream sequences such as promoters and regulatory elements. For example, restriction-site PCR uses universal primers to retrieve unknown sequence adjacent to a known locus (Sarkar, PCR Methods Applic. 2, 318-322, 1993). Genomic DNA is first amplified in the presence of a primer to a linker sequence and a primer specific to the known region. The amplified sequences are then subjected to a second round of PCR with the same linker primer and another specific primer internal to the first one. Products of each round of PCR are transcribed with an appropriate RNA polymerase and sequenced using reverse transcriptase.

Inverse PCR also can be used to amplify or extend sequences using divergent primers based on a known region (Triglia et al., Nucleic Acids Res. 16, 8186, 1988). Primers can be designed using commercially available software, such as OLIGO 4.06 Primer Analysis software (National Biosciences Inc., Plymouth, Minn.), to be 22-30 nucleotides in length, to have a GC content of 50% or more, and to anneal to the target sequence at temperatures about 68-72° C. The method uses several restriction enzymes to generate a suitable fragment in the known region of a gene. The fragment is then circularized by intramolecular ligation and used as a PCR template.

Another method which can be used is capture PCR, which involves PCR amplification of DNA fragments adjacent to a known sequence in human and yeast artificial chromosome DNA (Lagerstrom et al., PCR Methods Applic. 1, 111-119, 1991). In this method, multiple restriction enzyme digestions and ligations also can be used to place an engineered double-stranded sequence into an unknown fragment of the DNA molecule before performing PCR.

Another method which can be used to retrieve unknown sequences is that of Parker et al., Nucleic Acids Res. 19, 3055-3060, 1991). Additionally, PCR, nested primers, and PROMOTERFINDER libraries (CLONTECH, Palo Alto, Calif.) can be used to walk genomic DNA (CLONTECH, Palo Alto, Calif.). This process avoids the need to screen libraries and is useful in finding intron/exon junctions.

When screening for full-length cDNAs, it is preferable to use libraries that have been size-selected to include larger cDNAs. Randomly-primed libraries are preferable, in that they will contain more sequences which contain the 5′ regions of genes. Use of a randomly primed library may be especially preferable for situations in which an oligo d(T) library does not yield a full-length cDNA. Genomic libraries can be useful for extension of sequence into 5′ non-transcribed regulatory regions.

Commercially available capillary electrophoresis systems can be used to analyze the size or confirm the nucleotide sequence of PCR or sequencing products. For example, capillary sequencing can employ flowable polymers for electrophoretic separation, four different fluorescent dyes (one for each nucleotide) which are laser activated, and detection of the emitted wavelengths by a charge coupled device camera. Output/light intensity can be converted to electrical signal using appropriate software (e.g. GENOTYPER and Sequence NAVIGATOR, Perkin Elmer), and the entire process from loading of samples to computer analysis and electronic data display can be computer controlled. Capillary electrophoresis is especially preferable for the sequencing of small pieces of DNA that might be present in limited amounts in a particular sample.

Obtaining Polypeptides

Obesity-specific polypeptides can be obtained, for example, by purification from rat hypothalamus cells, by expression of obesity-specific polynucleotides, or by direct chemical synthesis.

Protein Purification

Obesity-specific polypeptides can be purified from any cell that expresses the obesity-specific polypeptide, including host cells that have been transfected with obesity-specific expression constructs. Hypothalamus from obese rats is a source of obesity-specific polypeptides. A purified obesity-specific polypeptide is separated from other compounds that normally associate with the obesity-specific polypeptide in the cell, such as certain proteins, carbohydrates, or lipids, using methods well-known in the art. Such methods include, but are not limited to, size exclusion chromatography, ammonium sulfate fractionation, ion exchange chromatography, affinity chromatography, and preparative gel electrophoresis. A preparation of purified obesity-specific polypeptides is at least 80% pure; preferably, the preparations are 90%, 95%, or 99% pure. Purity of the preparations can be assessed by any means known in the art, such as SDS-polyacrylamide gel electrophoresis.

Expression of Polynucleotides

To express an obesity-specific polynucleotide, the polynucleotide can be inserted into an expression vector that contains the necessary elements for the transcription and translation of the inserted coding sequence. Methods that are well known to those skilled in the art can be used to construct expression vectors containing sequences encoding obesity-specific polypeptides and appropriate transcriptional and translational control elements. These methods include in vitro recombinant DNA techniques, synthetic techniques, and in vivo genetic recombination. Such techniques are described, for example, in Sambrook et al. (1989) and in Ausubel et al., CURRENT PROTOCOLS IN MOLECULAR BIOLOGY, John Wiley & Sons, New York, N.Y., 1989.

A variety of expression vector/host systems can be utilized to contain and express sequences encoding an obesity-specific polypeptide. These include, but are not limited to, microorganisms, such as bacteria transformed with recombinant bacteriophage, plasmid, or cosmid DNA expression vectors; yeast transformed with yeast expression vectors, insect cell systems infected with virus expression vectors (e.g., baculovirus), plant cell systems transformed with virus expression vectors (e.g., cauliflower mosaic virus, CaMV; tobacco mosaic virus, TMV) or with bacterial expression vectors (e.g., Ti or pBR322 plasmids), or animal cell systems.

The control elements or regulatory sequences are those non-translated regions of the vector—enhancers, promoters, 5′ and 3′ untranslated regions—which interact with host cellular proteins to carry out transcription and translation. Such elements can vary in their strength and specificity. Depending on the vector system and host utilized, any number of suitable transcription and translation elements, including constitutive and inducible promoters, can be used. For example, when cloning in bacterial systems, inducible promoters such as the hybrid lacZ promoter of the BLUESCRIPT phagemid (Stratagene, LaJolla, Calif.) or pSPORT1 plasmid (Life Technologies) and the like can be used. The baculovirus polyhedrin promoter can be used in insect cells. Promoters or enhancers derived from the genomes of plant cells (e.g., heat shock, RUBISCO, and storage protein genes) or from plant viruses (e.g., viral promoters or leader sequences) can be cloned into the vector. In mammalian cell systems, promoters from mammalian genes or from mammalian viruses are preferable. If it is necessary to generate a cell line that contains multiple copies of a nucleotide sequence encoding an obesity-specific polypeptide, vectors based on SV40 or EBV can be used with an appropriate selectable marker.

Bacterial and Yeast Expression Systems

In bacterial systems, a number of expression vectors can be selected depending upon the use intended for the obesity-specific polypeptide. For example, when a large quantity of an obesity-specific polypeptide is needed for the induction of antibodies, vectors which direct high level expression of fusion proteins that are readily purified can be used. Such vectors include, but are not limited to, multifunctional E. coli cloning and expression vectors such as BLUESCRIPT (Stratagene). In a BLUESCRIPT vector, a sequence encoding the an obesity-specific polypeptide can be ligated into the vector in frame with sequences for the amino-terminal Met and the subsequent 7 residues of β-galactosidase so that a hybrid protein is produced. pIN vectors (Van Heeke & Schuster, J. Biol. Chem. 264, 5503-5509, 1989) or pGEX vectors (Promega, Madison, Wis.) also can be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems can be designed to include heparin, thrombin, or factor Xa protease cleavage sites so that the cloned polypeptide of interest can be released from the GST moiety at will.

In the yeast Saccharomyces cerevisiae, a number of vectors containing constitutive or inducible promoters such as alpha factor, alcohol oxidase, and PGH can be used. For reviews, see Ausubel et al. (1989) and Grant et al., Methods Enzymol. 153, 516-544, 1987.

Plant and Insect Expression Systems

If plant expression vectors are used, the expression of sequences encoding an obesity-specific polypeptide can be driven by any of a number of promoters. For example, viral promoters such as the 35S and 19S promoters of CaMV can be used alone or in combination with the omega leader sequence from TMV (Takamatsu, EMBO J. 6, 307-311, 1987). Alternatively, plant promoters such as the small subunit of RUBISCO or heat shock promoters can be used (Coruzzi et al., EMBO J. 3, 1671-1680, 1984; Broglie et al., Science 224, 838-843, 1984; Winter et al., Results Probl. Cell Differ. 17, 85-105, 1991). These constructs can be introduced into plant cells by direct DNA transformation or by pathogen-mediated transfection. Such techniques are described in a number of generally available reviews (e.g., Hobbs or Murray, in MCGRAW HILL YEARBOOK OF SCIENCE AND TECHNOLOGY, McGraw Hill, New York, N.Y., pp. 191-196,1992).

An insect system also can be used to express an obesity-specific polypeptide. For example, in one such system Autographa californica nuclear polyhedrosis virus (AcNPV) is used as a vector to express foreign genes in Spodoptera frugiperda cells or in Trichoplusia larvae. Sequences encoding an obesity-specific polypeptide can be cloned into a non-essential region of the virus, such as the polyhedrin gene, and placed under control of the polyhedrin promoter. Successful insertion of an obesity-specific polypeptide will render the polyhedrin gene inactive and produce recombinant virus lacking coat protein. The recombinant viruses can then be used to infect S. frugiperda cells or Trichoplusia larvae in which an obesity-specific polypeptide can be expressed (Engelhard et al., Proc. Nat. Acad. Sci. 91, 3224-3227, 1994).

Mammalian Expression Systems

A number of viral-based expression systems can be used to express obesity-specific polypeptides in mammalian host cells. For example, if an adenovirus is used as an expression vector, sequences encoding obesity-specific polypeptides can be ligated into an adenovirus transcription/translation complex comprising the late promoter and tripartite leader sequence. Insertion in a non-essential E1 or E3 region of the viral genome can be used to obtain a viable virus that is capable of expressing obesity-specific polypeptide in infected host cells (Logan & Shenk, Proc. Natl. Acad. Sci. 81, 3655-3659, 1984). If desired, transcription enhancers, such as the Rous sarcoma virus (RSV) enhancer, can be used to increase expression in mammalian host cells.

Human artificial chromosomes (HACs) also can be used to deliver larger fragments of DNA than can be contained and expressed in a plasmid. HACs of 6M to 10M are constructed and delivered to cells via conventional delivery methods (e.g., liposomes, polycationic amino polymers, or vesicles).

Specific initiation signals also can be used to achieve more efficient translation of sequences encoding obesity-specific polypeptides. Such signals include the ATG initiation codon and adjacent sequences. In cases where sequences encoding obesity-specific polypeptide, its initiation codon, and upstream sequences are inserted into the appropriate expression vector, no additional transcriptional or translational control signals may be needed. However, in cases where only coding sequence, or a fragment thereof, is inserted, exogenous translational control signals (including the ATG initiation codon) should be provided. The initiation codon should be in the correct reading frame to ensure translation of the entire insert. Exogenous translational elements and initiation codons can be of various origins, both natural and synthetic. The efficiency of expression can be enhanced by the inclusion of enhancers that are appropriate for the particular cell system which is used (see Scharf et al., Results Probl. Cell Differ. 20, 125-162, 1994).

Host Cells

A host cell strain can be chosen for its ability to modulate the expression of the inserted sequences or to process the expressed obesity-specific polypeptide in the desired fashion. Such modifications of the polypeptide include, but are not limited to, acetylation, carboxylation, glycosylation, phosphorylation, lipidation, and acylation. Post-translational processing that cleaves a “prepro” form of the polypeptide also can be used to facilitate correct insertion, folding and/or function. Different host cells that have specific cellular machinery and characteristic mechanisms for post-translational activities (e.g., CHO, HeLa, MDCK, HEK293, and WI38), are available from the American Type Culture Collection (ATCC; 10801 University Boulevard, Manassas, Va. 20110-2209) and can be chosen to ensure the correct modification and processing of the foreign protein.

Stable expression is preferred for long-term, high-yield production of recombinant proteins. For example, cell lines that stably express obesity-specific polypeptides can be transformed using expression vectors that can contain viral origins of replication and/or endogenous expression elements and a selectable marker gene on the same or on a separate vector. Following the introduction of the vector, cells can be allowed to grow for 1-2 days in an enriched medium before they are switched to a selective medium. The purpose of the selectable marker is to confer resistance to selection, and its presence allows growth and recovery of cells that successfully express the introduced obesity-specific sequences. Resistant clones of stably transformed cells can be proliferated using tissue culture techniques appropriate to the cell type. See, for example, ANIMAL CELL CULTURE, R. I. Freshney, ed., 1986.

Any number of selection systems can be used to recover transformed cell lines. These include, but are not limited to, the herpes simplex virus thymidine kinase (Wigler et al., Cell 11, 223-32, 1977) and adenine phosphoribosyltransferase (Lowy et al., Cell 22, 817-23, 1980) genes that can be employed in tk⁻ or aprt⁻ cells, respectively. Also, antimetabolite, antibiotic, or herbicide resistance can be used as the basis for selection. For example, dhfr confers resistance to methotrexate (Wigler et al., Proc. Natl. Acad. Sci. 77, 3567-70, 1980), npt confers resistance to the aminoglycosides, neomycin and G-418 (Colbere-Garapin et al., J. Mol. Biol. 150, 1-14, 1981), and als and pat confer resistance to chlorsulfuron and phosphinotricin acetyltransferase, respectively (Murray, 1992, supra). Additional selectable genes have been described. For example, trpB allows cells to utilize indole in place of tryptophan, or hisD, which allows cells to utilize histinol in place of histidine (Hartman & Mulligan, Proc. Natl. Acad. Sci. 85, 8047-51, 1988). Visible markers such as anthocyanins, β-glucuronidase and its substrate GUS, and luciferase and its substrate luciferin, can be used to identify transformants and to quantify the amount of transient or stable protein expression attributable to a specific vector system (Rhodes et al., Methods Mol. Biol. 55, 121-131, 1995).

Detecting Expression

Although the presence of marker gene expression suggests that the obesity-specific polynucleotide is also present, its presence and expression may need to be confirmed. For example, if a sequence encoding obesity-specific polypeptide is inserted within a marker gene sequence, transformed cells containing sequences that encode obesity-specific polypeptide can be identified by the absence of marker gene function. Alternatively, a marker gene can be placed in tandem with a sequence encoding an obesity-specific polypeptide under the control of a single promoter. Expression of the marker gene in response to induction or selection usually indicates expression of the obesity-specific polynucleotide.

Alternatively, host cells which contain an obesity-specific polynucleotide and which express an obesity-specific polypeptide can be identified by a variety of procedures known to those of skill in the art. These procedures include, but are not limited to, DNA-DNA or DNA-RNA hybridizations and protein bioassay or immunoassay techniques that include membrane, solution, or chip-based technologies for the detection and/or quantification of nucleic acid or protein. For example, the presence of a polynucleotide sequence encoding an obesity-specific polypeptide can be detected by DNA-DNA or DNA-RNA hybridization or amplification using probes or fragments or fragments of polynucleotides encoding an obesity-specific polypeptide. Nucleic acid amplification-based assays involve the use of oligonucleotides selected from sequences encoding an obesity-specific polypeptide to detect transformants that contain an obesity-specific polynucleotide.

A variety of protocols for detecting and measuring the expression of an obesity-specific polypeptide, using either polyclonal or monoclonal antibodies specific for the polypeptide, are known in the art. Examples include enzyme-linked immunosorbent assay (ELISA), radioimmunoassay (RIA), and fluorescence activated cell sorting (FACS). A two-site, monoclonal-based immunoassay using monoclonal antibodies reactive to two non-interfering epitopes on an obesity-specific polypeptide can be used, or a competitive binding assay can be employed. These and other assays are described in Hampton et al., SEROLOGICAL METHODS: A LABORATORY MANUAL, APS Press, St. Paul, Minn., 1990) and Maddox et al., J. Exp. Med. 158, 1211-1216, 1983).

A wide variety of labels and conjugation techniques are known by those skilled in the art and can be used in various nucleic acid and amino acid assays. Means for producing labeled hybridization or PCR probes for detecting sequences related to polynucleotides encoding obesity-specific polypeptides include oligolabeling, nick translation, end-labeling, or PCR amplification using a labeled nucleotide. Alternatively, sequences encoding obesity-specific polypeptide can be cloned into a vector for the production of an mRNA probe. Such vectors are known in the art, are commercially available, and can be used to synthesize RNA probes in vitro by addition of labeled nucleotides and an appropriate RNA polymerase such as T7, T3, or SP6. These procedures can be conducted using a variety of commercially available kits (Amersham Pharmacia Biotech, Promega, and US Biochemical). Suitable reporter molecules or labels which can be used for ease of detection include radionuclides, enzymes, and fluorescent, chemiluminescent, or chromogenic agents, as well as substrates, cofactors, inhibitors, magnetic particles, and the like.

Expression and Purification of Polypeptides

Host cells transformed with nucleotide sequences encoding obesity-specific polypeptide can be cultured under conditions suitable for the expression and recovery of the protein from cell culture. The polypeptide produced by a transformed cell can be secreted or contained intracellularly depending on the sequence and/or the vector used. As will be understood by those of skill in the art, expression vectors containing polynucleotides which encode obesity-specific polypeptides can be designed to contain signal sequences which direct secretion of soluble obesity-specific polypeptides through a prokaryotic or eukaryotic cell membrane or which direct the membrane insertion of membrane-bound obesity-specific polypeptide.

As discussed above, other constructions can be used to join a sequence encoding an obesity-specific polypeptide to a nucleotide sequence encoding a polypeptide domain that will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp., Seattle, Wash.). Inclusion of cleavable linker sequences such as those specific for Factor Xa or enterokinase (Invitrogen, San Diego, Calif.) between the purification domain and the obesity-specific polypeptide also can be used to facilitate purification. One such expression vector provides for expression of a fusion protein containing an obesity-specific polypeptide and 6 histidine residues preceding a thioredoxin or an enterokinase cleavage site. The histidine residues facilitate purification by IMAC (immobilized metal ion affinity chromatography, as described in Porath et al., Prot. Exp. Purif. 3, 263-281, 1992), while the enterokinase cleavage site provides a means for purifying the obesity-specific polypeptide from the fusion protein. Vectors that contain fusion proteins are disclosed in Kroll et al., DNA Cell Biol. 12, 441-453, 1993.

Chemical Synthesis

Sequences encoding an obesity-specific polypeptide can be synthesized, in whole or in part, using chemical methods well known in the art (see Caruthers et al., Nucl. Acids Res. Symp. Ser. 215-223, 1980; Horn et al. Nucl. Acids Res. Symp. Ser. 225-232, 1980). Alternatively, an obesity-specific polypeptide itself can be produced using chemical methods to synthesize its amino acid sequence, such as by direct peptide synthesis using solid-phase techniques (Merrifield, J. Am. Chem. Soc. 85, 2149-2154, 1963; Roberge et al., Science 269, 202-204, 1995). Protein synthesis can be performed using manual techniques or by automation. Automated synthesis can be achieved, for example, using Applied Biosystems 431A Peptide Synthesizer (Perkin Elmer). Optionally, fragments of obesity-specific polypeptides can be separately synthesized and combined using chemical methods to produce full-length molecules.

The newly synthesized peptide can be substantially purified by preparative high performance liquid chromatography (e.g., Creighton, PROTEINS: STRUCTURES AND MOLECULAR PRINCIPLES, W H Freeman and Co., New York, N.Y., 1983). The composition of a synthetic obesity-specific polypeptide can be confirmed by amino acid analysis or sequencing (e.g., the Edman degradation procedure; see Creighton, supra). Additionally, any portion of the amino acid sequence of the obesity-specific polypeptide can be altered during direct synthesis and/or combined using chemical methods with sequences from other proteins to produce a variant polypeptide or a fusion protein.

Production of Altered Polypetides

As will be understood by those of skill in the art, it may be advantageous to produce obesity-specific polypeptide-encoding nucleotide sequences possessing non-naturally occurring codons. For example, codons preferred by a particular prokaryotic or eukaryotic host can be selected to increase the rate of protein expression or to produce an RNA transcript having desirable properties, such as a half-life that is longer than that of a transcript generated from the naturally occurring sequence.

The nucleotide sequences disclosed herein can be engineered using methods generally known in the art to alter obesity-specific polypeptide-encoding sequences for a variety of reasons, including but not limited to, alterations which modify the cloning, processing, and/or expression of the polypeptide or mRNA product. DNA shuffling by random fragmentation and PCR reassembly of gene fragments and synthetic oligonucleotides can be used to engineer the nucleotide sequences. For example, site-directed mutagenesis can be used to insert new restriction sites, alter glycosylation patterns, change codon preference, produce splice variants, introduce mutations, and so forth.

Antibodies

Any type of antibody known in the art can be generated to bind specifically to an epitope of an obesity-specific polypeptide. “Antibody” as used herein includes intact immunoglobulin molecules, as well as fragments thereof, such as Fab, F(ab′)₂, and Fv, which are capable of binding an epitope of an obesity-specific polypeptide. Typically, at least 6, 8, 10, or 12 contiguous amino acids are required to form an epitope. However, epitopes which involve non-contiguous amino acids may require more, e.g., at least 15, 25, or 50 amino acids.

An antibody which specifically binds to an epitope of an obesity-specific polypeptide can be used therapeutically, as well as in immunochemical assays, such as Western blots, ELISAs, radioimmunoassays, immunohistochemical assays, immunoprecipitations, or other immunochemical assays known in the art. Various immunoassays can be used to identify antibodies having the desired specificity. Numerous protocols for competitive binding or immunoradiometric assays are well known in the art. Such immunoassays typically involve the measurement of complex formation between an immunogen and an antibody that specifically binds to the immunogen.

Typically, an antibody that specifically binds to an obesity-specific polypeptide provides a detection signal at least 5-, 10-, or 20-fold higher than a detection signal provided with other proteins when used in an immunochemical assay. Preferably, antibodies that specifically bind to obesity-specific polypeptides do not detect other proteins in immunochemical assays and can immunoprecipitate an obesity-specific polypeptide from solution.

Obesity-specific polypeptides can be used to immunize a mammal, such as a mouse, rat, rabbit, guinea pig, monkey, or human, to produce polyclonal antibodies. If desired, an obesity-specific polypeptide can be conjugated to a carrier protein, such as bovine serum albumin, thyroglobulin, and keyhole limpet hemocyanin. Depending on the host species, various adjuvants can be used to increase the immunological response. Such adjuvants include, but are not limited to, Freund's adjuvant, mineral gels (e.g., aluminum hydroxide), and surface-active substances (e.g. lysolecithin, pluronic polyols, polyanions, peptides, oil emulsions, keyhole limpet hemocyanin, and dinitrophenol). Among adjuvants used in humans, BCG (bacilli Calmette-Guerin) and Corynebacterium parvum are especially useful.

Monoclonal antibodies that specifically bind to an obesity-specific polypeptide can be prepared using any technique which provides for the production of antibody molecules by continuous cell lines in culture. These techniques include, but are not limited to, the hybridoma technique, the human B-cell hybridoma technique, and the EBV-hybridoma technique (Kohler et al., Nature 256, 495-497, 1985; Kozbor et al., J. Immunol. Methods 81, 31-42, 1985; Cote et al., Proc. Natl. Acad. Sci. 80, 2026-2030, 1983; Cole et al., Mol. Cell Biol. 62, 109-120, 1984).

In addition, techniques developed for the production of “chimeric antibodies,” the splicing of mouse antibody genes to human antibody genes to obtain a molecule with appropriate antigen specificity and biological activity, can be used (Morrison et al., Proc. Natl. Acad. Sci. 81, 6851-6855, 1984; Neuberger et al., Nature 312, 604-608, 1984; Takeda et al., Nature 314, 452-454, 1985). Monoclonal and other antibodies also can be “humanized” to prevent a patient from mounting an immune response against the antibody when it is used therapeutically. Such antibodies may be sufficiently similar in sequence to human antibodies to be used directly in therapy or may require alteration of a few key residues. Sequence differences between rodent antibodies and human sequences can be minimized by replacing residues which differ from those in the human sequences by site directed mutagenesis of individual residues or by grating of entire complementarity determining regions. Alternatively, humanized antibodies can be produced using recombinant methods, as described in GB2188638B. Antibodies that specifically bind to an obesity-specific polypeptide can contain antigen binding sites which are either partially or fully humanized, as disclosed in U.S. Pat. No. 5,565,332.

Alternatively, techniques described for the production of single chain antibodies can be adapted using methods known in the art to produce single chain antibodies that specifically bind to obesity-specific polypeptides. Antibodies with related specificity, but of distinct idiotypic composition, can be generated by chain shuffling from random combinatorial immunoglobin libraries (Burton, Proc. Natl. Acad. Sci. 88, 11120-23, 1991).

Single-chain antibodies also can be constructed using a DNA amplification method, such as PCR, using hybridoma cDNA as a template (Thirion et al., 1996, Eur. J. Cancer Prev. 5, 507-11). Single-chain antibodies can be mono- or bispecific, and can be bivalent or tetravalent. Construction of tetravalent, bispecific single-chain antibodies is taught, for example, in Coloma & Morrison, 1997, Nat. Biotechnol. 15, 159-63. Construction of bivalent, bispecific single-chain antibodies is taught in Mallender & Voss, 1994, J. Biol. Chem. 269, 199-206.

A nucleotide sequence encoding a single-chain antibody can be constructed using manual or automated nucleotide synthesis, cloned into an expression construct using standard recombinant DNA methods, and introduced into a cell to express the coding sequence, as described below. Alternatively, single-chain antibodies can be produced directly using, for example, filamentous phage technology (Verhaar et al., 1995, Int. J. Cancer 61, 497-501; Nicholls et al., 1993, J. Immunol. Meth. 165, 81-91).

Antibodies which specifically bind to obesity-specific polypeptides also can be produced by inducing in vivo production in the lymphocyte population or by screening immunoglobulin libraries or panels of highly specific binding reagents as disclosed in the literature (Orlandi et al., Proc. Natl. Acad. Sci. 86, 3833-3837, 1989; Winter et al., Nature 349, 293-299, 1991).

Other types of antibodies can be constructed and used therapeutically in methods of the invention. For example, chimeric antibodies can be constructed as disclosed in WO 93/03151. Binding proteins which are derived from immunoglobulins and which are multivalent and multispecific, such as the “diabodies” described in WO 94/13804, also can be prepared.

Antibodies according to the invention can be purified by methods well known in the art. For example, antibodies can be affinity purified by passage over a column to which an obesity-specific polypeptide is bound. The bound antibodies can then be eluted from the column using a buffer with a high salt concentration.

Antisense Oligonucleotides

Antisense oligonucleotides are nucleotide sequences that are complementary to a specific DNA or RNA sequence. Once introduced into a cell, the complementary nucleotides combine with natural sequences produced by the cell to form complexes and block either transcription or translation. Preferably, an antisense oligonucleotide is at least 11 nucleotides in length, but can be at least 12, 15, 20, 25, 30, 35, 40, 45, or 50 or more nucleotides long. Longer sequences also can be used. Antisense oligonucleotide molecules can be provided in a DNA construct and introduced into a cell as described above to decrease the level of obesity-specific gene products in the cell.

Antisense oligonucleotides can be deoxyribonucleotides, ribonucleotides, or a combination of both. Oligonucleotides can be synthesized manually or by an automated synthesizer, by covalently linking the 5′ end of one nucleotide with the 3′ end of another nucleotide with non-phosphodiester intemucleotide linkages such alkylphosphonates, phosphorothioates, phosphorodithioates, alkylphosphonothioates, alkylphosphonates, phosphoramidates, phosphate esters, carbamates, acetamidate, carboxymethyl esters, carbonates, and phosphate triesters. See Brown, Meth. Mol. Biol. 20, 1-8, 1994; Sonveaux, Meth. Mol. Biol. 26, 1-72, 1994; Uhlmann et al., Chem. Rev. 90, 543-583, 1990.

Modifications of obesity-specific gene expression can be obtained by designing antisense oligonucleotides that will form duplexes to the control, 5′, or regulatory regions of the obesity-specific gene. Oligonucleotides derived from the transcription initiation site, e.g., between positions −10 and +10 from the start site, are preferred. Similarly, inhibition can be achieved using “triple helix” base-pairing methodology. Triple helix pairing is useful because it causes inhibition of the ability of the double helix to open sufficiently for the binding of polymerases, transcription factors, or chaperons. Therapeutic advances using triplex DNA have been described in the literature (e.g., Gee et al., in Huber & Carr, MOLECULAR AND IMMUNOLOGIC APPROACHES, Futura Publishing Co., Mt. Kisco, N.Y., 1994). An antisense oligonucleotide also can be designed to block translation of mRNA by preventing the transcript from binding to ribosomes.

Precise complementarity is not required for successful complex formation between an antisense oligonucleotide and the complementary sequence of an obesity-specific polynucleotide. Antisense oligonucleotides that comprise, for example, 2, 3, 4, or 5 or more stretches of contiguous nucleotides that are precisely complementary to an obesity-specific polynucleotide, each separated by a stretch of contiguous nucleotides which are not complementary to adjacent obesity-specific nucleotides, can provide sufficient targeting specificity for obesity-specific mRNA. Preferably, each stretch of complementary contiguous nucleotides is at least 4, 5, 6, 7, or 8 or more nucleotides in length. Non-complementary intervening sequences are preferably 1, 2, 3, or 4 nucleotides in length. One skilled in the art can easily use the calculated melting point of an antisense-sense pair to determine the degree of mismatching which will be tolerated between a particular antisense oligonucleotide and a particular obesity-specific polynucleotide sequence.

Antisense oligonucleotides can be modified without affecting their ability to hybridize to an obesity-specific polynucleotide. These modifications can be internal or at one or both ends of the antisense molecule. For example, internucleoside phosphate linkages can be modified by adding cholesteryl or diamine moieties with varying numbers of carbon residues between the amino groups and terminal ribose. Modified bases and/or sugars, such as arabinose instead of ribose, or a 3′, 5′-substituted oligonucleotide in which the 3′ hydroxyl group or the 5′ phosphate group are substituted, also can be employed in a modified antisense oligonucleotide. These modified oligonucleotides can be prepared by methods well known in the art. See, e.g., Agrawal et al., Trends Biotechnol. 10, 152-158, 1992; Uhlmann et al., Chem. Rev. 90, 543-584, 1990; Uhlmann et al., Tetrahedron. Lett. 215, 3539-3542, 1987.

Ribozymes

Ribozymes are RNA molecules with catalytic activity. See, e.g., Cech, Science 236, 1532-1539; 1987; Cech, Ann. Rev. Biochem. 59, 543-568; 1990, Cech, Curr. Opin. Struct. Biol. 2, 605-609; 1992, Couture & Stinchcomb, Trends Genet. 12, 510-515, 1996. Ribozymes can be used to inhibit gene function by cleaving an RNA sequence, as is known in the art (e.g., Haseloff et al., U.S. Pat. No. 5,641,673). The mechanism of ribozyme action involves sequence-specific hybridization of the ribozyme molecule to complementary target RNA, followed by endonucleolytic cleavage. Examples include engineered hammerhead motif ribozyme molecules that can specifically and efficiently catalyze endonucleolytic cleavage of specific nucleotide sequences.

The coding sequence of an obesity-specific polynucleotide can be used to generate ribozymes which will specifically bind to mRNA transcribed from an obesity-specific polynucleotide. Methods of designing and constructing ribozymes which can cleave other RNA molecules in trans in a highly sequence specific manner have been developed and described in the art (see Haseloff et al. Nature 334, 585-591, 1988). For example, the cleavage activity of ribozymes can be targeted to specific RNAs by engineering a discrete “hybridization” region into the ribozyme. The hybridization region contains a sequence complementary to the target RNA and thus specifically hybridizes with the target (see, for example, Gerlach et al., EP 321,201).

Specific ribozyme cleavage sites within an obesity-specific RNA target can be identified by scanning the target molecule for ribozyme cleavage sites which include the following sequences: GUA, GUU, and GUC. Once identified, short RNA sequences of between 15 and 20 ribonucleotides corresponding to the region of the target RNA containing the cleavage site can be evaluated for secondary structural features which may render the target inoperable. Suitability of candidate obesity-specific RNA targets also can be evaluated by testing accessibility to hybridization with complementary oligonucleotides using ribonuclease protection assays. Longer complementary sequences can be used to increase the affinity of the hybridization sequence for the target. The hybridizing and cleavage regions of the ribozyme can be integrally related such that upon hybridizing to the target RNA through the complementary regions, the catalytic region of the ribozyme can cleave the target.

Ribozymes can be introduced into cells as part of a DNA construct. Mechanical methods, such as microinjection, liposome-mediated transfection, electroporation, or calcium phosphate precipitation, can be used to introduce a ribozyme-containing DNA construct into cells in which it is desired to decrease obesity-specific gene expression. Alternatively, if it is desired that the cells stably retain the DNA construct, the construct can be supplied on a plasmid and maintained as a separate element or integrated into the genome of the cells, as is known in the art. A ribozyme-encoding DNA construct can include transcriptional regulatory elements, such as a promoter element, an enhancer or UAS element, and a transcriptional terminator signal, for controlling transcription of ribozymes in the cells.

As taught in Haseloff et al., U.S. Pat. No. 5,641,673, ribozymes can be engineered so that ribozyme expression will occur in response to factors that induce expression of a target gene. Ribozymes also can be engineered to provide an additional level of regulation, so that destruction of mRNA occurs only when both a ribozyme and a target gene are induced in the cells.

Identification of Target and Pathway Genes and Proteins

Described herein are methods for the identification of genes which are involved in body weight disorder states, and/or which are involved in appetite and body weight regulation. Such genes may represent genes that are differentially expressed in body weight disorder states relative to their expression in normal, or non-body weight disorder states. Further, such genes may represent genes that are differentially regulated in response to manipulations relevant to appetite and body weight regulation. Such differentially expressed genes may represent “target” and/or “fingerprint” genes. Methods for the identification of such differentially expressed genes are described below. Methods for the further characterization of such differentially expressed genes, and for their identification as target and/or fingerprint genes also are described below.

In addition, methods are described for the identification of genes, termed “pathway genes,” which are involved in body weight disorder states, and/or in appetite or body weight regulation. “Pathway gene,” as used herein, refers to a gene whose gene product exhibits the ability to interact with gene products involved in body weight disorders and/or to interact with gene products that are relevant to appetite or body weight regulation. A pathway gene may be differentially expressed and, therefore, may have the characteristics of a target and/or fingerprint gene.

“Differential expression” refers to both quantitative as well as qualitative differences in a gene's temporal and/or tissue expression pattern. Thus, a differentially expressed gene may qualitatively have its expression activated or completely inactivated in normal versus body weight disorder states, or under control versus experimental conditions. Such a qualitatively regulated gene will exhibit an expression pattern within a given tissue or cell type that is detectable in either control or body weight disorder subjects, but is not detectable in both. Alternatively, such a qualitatively regulated gene will exhibit an expression pattern within a given tissue or cell type that is detectable in either control or experimental subjects, but is not detectable in both. “Detectable” refers to an RNA expression pattern which is detectable via the standard techniques of differential display, RT-PCR and/or Northern analyses, which are well known to those of skill in the art.

A differentially expressed gene may have its expression modulated, i.e., quantitatively increased or decreased, in normal versus body weight disorder states, or under control versus experimental conditions. The degree to which expression differs in normal versus body weight disorder or control versus experimental states need only be large enough to be visualized via standard characterization techniques, such as, for example, the differential display technique described below. Other such standard characterization techniques by which expression differences may be visualized include but are not limited to, quantitative RT (reverse transcriptase) PCR and Northern analyses.

Differentially expressed genes may be further described as target genes and/or fingerprint genes. “Fingerprint gene” refers to a differentially expressed gene whose expression pattern may be utilized as part of a prognostic or diagnostic body weight disorder evaluation, or which, alternatively, may be used in methods for identifying compounds useful for the treatment of body weight disorders. A fingerprint gene may also have the characteristics of a target gene or a pathway gene.

“Target gene” refers to a differentially expressed gene involved in body weight disorders and/or appetite or body regulation in a manner by which modulation of the level of target gene expression or of target gene product activity may act to ameliorate symptoms of body weight disorders including, but are not limited to, obesity. A target gene may also have the characteristics of a fingerprint gene and/or a pathway gene.

Identification of Differentially Expressed Genes

A variety of methods may be utilized for the identification of genes which are involved in body weight disorder states, and/or which are involved in appetite and body weight regulation. Among the paradigms which may be utilized for the identification of differentially expressed genes involved in, for example, body weight disorders, are paradigms designed to analyze those genes which may be involved in short term appetite control. Accordingly, such paradigms are referred to as “short term appetite control paradigms.” These paradigms may serve to identify genes involved in signaling hunger and satiety.

In one embodiment of such a paradigm, test subjects, preferably mice, may be fed normally prior to the initiation of the paradigm study, then divided into one control and two experimental groups. The control group would then be maintained on ad lib nourishment, while the first experimental group (“fasted group”) would be fasted, and the second experimental group (“fasted-refed group”) would initially be fasted, and would then be offered a highly palatable meal shortly before the collection of tissue samples. Each test animal should be weighted immediately prior to and immediately after the experiment. Among additional paradigms which may be utilized for the identification of differentially expressed genes involved in, for example, body weight disorders, are paradigms designed to analyze those genes which may be involved genetic obesity. Accordingly, such paradigms are referred to as “genetic obesity paradigms.” In the case of mice, for example, such paradigms may identify genes regulated by the ob, db, and/or tub gene products. In the case of rats, for example, such paradigms may identify genes regulated by the fatty (fa) gene product. In one embodiment of such a paradigm, test subjects may include ob/ob, db/db, and/or tub/tub experimental mice and lean littermate control animals. Such animals would be offered normal nourishment for a given period, after which tissue samples would be collected for analysis. In additional embodiments, ob/ob, db/db, and/or tub/tub experimental mice and lean control animals may be utilized as part of the short term appetite control paradigms discussed above, or as part of the set point and/or drug study paradigms discussed below.

Paradigms which may be utilized for the identification of differentially expressed genes involved in body weight disorders may include paradigms designed to identify those genes which may be regulated in response to changes in body weight. Such paradigms may be referred to as “set point paradigms.” In one embodiment of such a paradigm, test subjects, preferably mice, may be fed normally prior to the initiation of the paradigm study, then divided into one control and two experimental groups. The control group would then be maintained on an ad lib diet of normal nourishment in order to calculate daily food intake. The first experimental group (“underweight group”) would then be underfed by receiving some fraction of normal food intake, 60-90% of normal, for example, so as to reduce and maintain the group's body weight to some percentage, for example 80%, of the control group. The second experimental group (“overweight group”) would be overfed by receiving a diet that would bring the group to some level above that of the control, for example 125% of the control group. Tissue samples would then be obtained for analysis. Human subjects may be utilized for the identification of obesity-associated genes. In one embodiment of such a paradigm, tissue samples may be obtained from obese and lean human subjects and analyzed for the presence of genes which are differentially expressed in the tissue of one group as opposed to another (e.g. differentially expressed in lean versus obese subjects). In another embodiment, obese human subjects may be studied over the course of a period of weight loss, achieved through food restriction. Tissue from these previously obese subjects may be analyzed for differential expression of gene products relative to tissue obtained from control (lean, non-previously obese) and obese subjects.

Paradigms may be utilized for the identification of differentially expressed genes involved in body weight disorders may additionally include paradigms designed to identify genes associated with body weight disorders induced by some physical manipulation to the test subject, such as, for example, hypothalamic lesion-induced body weight disorders. For example, bilateral lesions in the ventromedial hypothalamus (VMH) of rodents may be utilized to induce hyperphagia and gross obesity in test subjects, while bilateral lesions in the ventrolateral hypothalamus (VLH) of rodents may be utilized to induce aphagia in test subjects. In such paradigms, tissue from hypothalamic-lesioned test subjects and from control subjects would be analyzed for the identification of genes that are differentially expressed in control versus lesioned animals.

Drugs known to affect human or animal body weight and/or appetite, such as short-term appetite, may be incorporated into paradigms designed to identify genes that are involved in body weight disorders and/or body weight or appetite regulation. Such paradigms are referred to as “drug study paradigms.” Such compounds may include known therapeutics, as well as compounds that are not useful as therapeutics due to, for example, their harmful side effects. Among the categories of control and test subjects which may be utilized in such paradigms are, for example, lean subjects, obese subjects, and obese subjects which have received the drug of interest. In various embodiments of the paradigms, subjects such as these may be fed a normal ad lib diet, a caloric restriction maintained diet, or a caloric restriction ad lib diet. Control and test subjects may additionally be pairfed, i.e., the control and test subjects may be fed via a coupled feeding device such that both control and test subjects receive identical amounts and types of food).

To identify differentially expressed genes, RNA, either total or mRNA, may be isolated from one or more tissues of the subjects utilized in paradigms such as those described above. RNA samples are obtained from tissues of experimental subjects and from corresponding tissues of control subjects. Any RNA isolation technique that does not select against the isolation of mRNA may be utilized for the purification of such RNA samples. See, for example, Ausubel et al., eds., CURRENT PROTOCOLS IN MOLECULAR BIOLOGY, John Wiley & Sons, Inc. New York, 1987-1993. Large numbers of tissue samples may readily be processed using techniques well known to those of skill in the art, such as, for example, the single-step RNA isolation process of Chomczynski, U.S. Pat. No. 4,843,155.

Transcripts within the collected RNA samples that represent RNA produced by differentially expressed genes may be identified by utilizing a variety of methods that are well known to those of skill in the art. For example, differential screening (Tedder et al., Proc. Natl. Acad. Sci. U.S.A. 85, 208-12, 1988), subtractive hybridization (Hedrick et al., Nature 308, 149-53; Lee et al., Proc. Natl. Acad. Sci. U.S.A. 88, 2825, 1984), and, preferably, differential display (Liang & Pardee, Science 257, 967-71, 1992; U.S. Pat. No. 5,262,311), may be utilized to identify nucleic acid sequences derived from genes that are differentially expressed.

Differential screening involves the duplicate screening of a cDNA library in which one copy of the library is screened with a total cell cDNA probe corresponding to the mRNA population of one cell type while a duplicate copy of the cDNA library is screened with a total cDNA probe corresponding to the mRNA population of a second cell type. For example, one cDNA probe may correspond to a total cell cDNA probe of a cell type or tissue derived from a control subject, while the second cDNA probe may correspond to a total cell cDNA probe of the same cell type or tissue derived from an experimental subject. Those clones that hybridize to one probe but not to the other potentially represent clones derived from genes differentially expressed in the cell type of interest in control versus experimental subjects.

Subtractive hybridization techniques generally involve the isolation of mRNA taken from two different sources, e.g., control and experimental tissue or cell type, the hybridization of the mRNA or single-stranded cDNA reverse-transcribed from the isolated mRNA, and the removal of all hybridized, and therefore double-stranded, sequences. The remaining non-hybridized, single-stranded cDNAs, potentially represent clones derived from genes that are differentially expressed in the two mRNA sources. Such single-stranded cDNAs are then used as the starting material for the construction of a library comprising clones derived from differentially expressed genes.

The differential display technique describes a procedure, utilizing the well-known polymerase chain reaction (PCR; the experimental embodiment set forth in Mullis, U.S. Pat. No. 4,683,202), which allows for the identification of sequences derived from genes that are differentially expressed. First, isolated RNA is reverse-transcribed into single-stranded cDNA, utilizing standard techniques that are well known to those of skill in the art. Primers for the reverse transcriptase reaction may include, but are not limited to, oligo dT-containing primers.

Next, this technique uses pairs of PCR primers, as described below, which allow for the amplification of clones representing a random subset of the RNA transcripts present within any given cell. Utilizing different pairs of primers allows each of the mRNA transcripts present in a cell to be amplified. Among such amplified transcripts may be identified those which have been produced from differentially expressed genes.

The 3′ oligonucleotide primer of the primer pairs may contain an oligo dT stretch of 10-13, preferably 11, dT nucleotides at its 5′ end, which hybridizes to the poly(A) tail of mRNA or to the complement of a cDNA reverse transcribed from an MRNA poly(A) tail. Second, in order to increase the specificity of the 3′ primer, the primer may contain one or more, preferably two, additional nucleotides at its 3′ end. Because, statistically, only a subset of the mRNA derived sequences present in the sample of interest will hybridize to such primers, the additional nucleotides allow the primers to amplify only a subset of the mRNA-derived sequences present in the sample of interest. This is preferred in that it allows more accurate and complete visualization and characterization of each of the bands representing amplified sequences.

The 5′ primer may contain a nucleotide sequence expected, statistically, to have the ability to hybridize to cDNA sequences derived from the tissues of interest. The nucleotide sequence may be an arbitrary one, and the length of the 5′ oligonucleotide primer may range from about 9 to about 15 nucleotides, with about 13 nucleotides being preferred. Arbitrary primer sequences cause the lengths of the amplified partial cDNAs produced to be variable, thus allowing different clones to be separated by using standard denaturing sequencing gel electrophoresis.

PCR reaction conditions should be chosen which optimize amplified product yield and specificity, and, additionally, produce amplified products of lengths, which may be resolved utilizing standard gel electrophoresis techniques. Such reaction conditions are well known to those of skill in the art, and important reaction parameters include, for example, length and nucleotide sequence of oligonucleotide primers as discussed above, and annealing and elongation step temperatures and reaction times.

The pattern of clones resulting from the reverse transcription and amplification of the mRNA of two different cell types is displayed via sequencing gel electrophoresis and compared. Differentially expressed genes are indicated by differences in the two banding patterns.

Once potentially differentially expressed gene sequences have been identified via bulk techniques such as, for example, those described above, the differential expression of such putatively differentially expressed genes should be corroborated. Corroboration may be accomplished via, for example, such well-known techniques as Northern analysis, quantitative RT PCR or RNase protection. Upon corroboration, the differentially expressed genes may be further characterized, and may be identified as target and/or fingerprint genes, as discussed below.

Amplified sequences of differentially expressed genes obtained through, for example, differential display may be used to isolate full-length clones of the corresponding gene. The full-length coding portion of the gene may readily be isolated, without undue experimentation, by molecular biological techniques well known in the art. For example, the isolated differentially expressed amplified fragment may be labeled and used to screen a cDNA library. Alternatively, the labeled fragment may be used to screen a genomic library.

PCR technology may also be utilized to isolate full-length cDNA sequences. As described above, the isolated, amplified gene fragments obtained through differential display have 5′ terminal ends at some random point within the gene and usually have 3′ terminal ends at a position corresponding to the 3′ end of the transcribed portion of the gene. Once nucleotide sequence information from an amplified fragment is obtained, the remainder of the gene (i.e., the 5′ end of the gene, when utilizing differential display) may be obtained using, for example, RT-PCR.

In one embodiment of such a procedure for the identification and cloning of full-length gene sequences, RNA may be isolated, following standard procedures, from an appropriate tissue or cellular source. A reverse transcription reaction may then be performed on the RNA using an oligonucleotide primer complimentary to the mRNA that corresponds to the amplified fragment, for the priming of first strand synthesis. Because the primer is anti-parallel to the mRNA, extension will proceed toward the 5′ end of the mRNA. The resulting RNA/DNA hybrid may then be “tailed” with guanines using a standard terminal transferase reaction, the hybrid may be digested with RNAase H, and second strand synthesis may then be primed with a poly-C primer. Using the two primers, the 5′ portion of the gene is amplified using PCR. Sequences obtained may then be isolated and recombined with previously isolated sequences to generate a full-length cDNA of the differentially expressed genes of the invention. For a review of cloning strategies and recombinant DNA techniques, see e.g., Sambrook et al., 1989, and Ausubel et al., 1989.

Identification of Pathway Genes

Methods are described herein for the identification of pathway genes. “Pathway gene” refers to a gene whose gene product exhibits the ability to interact with gene products involved in body weight disorders and/or to interact with gene products that are relevant to appetite or body weight regulation. A pathway gene may be differentially expressed and, therefore, may have the characteristics of a target and/or fingerprint gene.

Any method suitable for detecting protein-protein interactions may be employed for identifying pathway gene products by identifying interactions between gene products and gene products known to be involved in body weight disorders and/or involved in appetite or body regulation. Such known gene products may be cellular or extracellular proteins. Those gene products that interact with such known gene products represent pathway gene products and the genes that encode them represent pathway genes.

Among the traditional methods that may be employed are co-immunoprecipitation, crosslinking and co-purification through gradients or chromatographic columns. Utilizing procedures such as these allows for the identification of pathway gene products. Once identified, a pathway gene product may be used, in conjunction with standard techniques, to identify its corresponding pathway gene. For example, at least a portion of the amino acid sequence of the pathway gene product may be ascertained using techniques well known to those of skill in the art, such as via the Edman degradation technique (see, e.g., Creighton, PROTEINS: STRUCTURES AND MOLECULAR PRINCIPLES, W. H. Freeman & Co., N.Y., pp.34-49, 1983). The amino acid sequence obtained may be used as a guide for the generation of oligonucleotide mixtures that can be used to screen for pathway gene sequences. Screening made be accomplished, for example, by standard hybridization or PCR techniques. Techniques for the generation of oligonucleotide mixtures and the screening are well-known. See, e.g., Ausubel, 1989, and Innis et al., eds., PCR PROTOCOLS: A GUIDE TO METHODS AND APPLICATIONS, 1990, Academic Press, Inc., New York.

Methods may be employed which result in the simultaneous identification of pathway genes which encode the protein interacting with a protein involved in body weight disorder states and/or appetite and body weight regulation. These methods include, for example, probing expression libraries with labeled protein known or suggested to be involved in body weight disorders and/or appetite or body weight regulation, using this protein in a manner similar to the well known technique of antibody probing of λgt11 libraries.

One method that detects protein interactions in vivo, the two-hybrid system, is described in detail for illustration only and not by way of limitation. One version of this system has been described in Chien et al., 1991, Proc. Natl. Acad. Sci. U.S.A. 88, 9578-82, 1991, and is commercially available from Clontech (Palo Alto, Calif.). Briefly, utilizing such a system, plasmids are constructed that encode two hybrid proteins: one consists of the DNA-binding domain of a transcription activator protein fused to a known protein, in this case, a protein known to be involved in body weight disorders and or processes relevant to appetite and/or weight regulation, and the other consists of the transcription activator protein's activation domain fused to an unknown protein that is encoded by a cDNA which has been recombined into this plasmid as part of a cDNA library. The plasmids are transformed into a strain of the yeast Saccharomyces cerevisiae that contains a reporter gene (e.g., lacZ) whose regulatory region contains the transcription activator's binding sites. Either hybrid protein alone cannot activate transcription of the reporter gene: the DNA-binding domain hybrid cannot because it does not provide activation function and the activation domain hybrid cannot because it cannot localize to the activator's binding sites. Interaction of the two hybrid proteins reconstitutes the functional activator protein and results in expression of the reporter gene, which is detected by an assay for the reporter gene product.

The two-hybrid system or related methodology may be used to screen activation domain libraries for proteins that interact with a known “bait” gene product. By way of example, and not by way of limitation, gene products known to be involved in body weight disorders and/or appetite or body weight regulation may be used as the bait gene products. These include but are not limited to the intracellular domain of receptors for such hormones as neuropeptide Y, galanin, interostatin, insulin, and CCK. Total genomic or cDNA sequences are fused to the DNA encoding an activation domain. This library and a plasmid encoding a hybrid of the bait gene product fused to the DNA-binding domain are cotransformed into a yeast reporter strain, and the resulting transformants are screened for those that express the reporter gene. For example, and not by way of limitation, the bait gene can be cloned into a vector such that it is translationally fused to the DNA encoding the DNA-binding domain of the GAL4 protein. These colonies are purified and the library plasmids responsible for reporter gene expression are isolated. DNA sequencing is then used to identify the proteins encoded by the library plasmids.

A cDNA library of the cell line from which proteins that interact with bait gene product are to be detected can be made using methods routinely practiced in the art. According to the particular system described herein, for example, the cDNA fragments can be inserted into a vector such that they are translationally fused to the activation domain of GAL4. This library can be co-transformed along with the bait gene-GAL4 fusion plasmid into a yeast strain that contains a lacZ gene driven by a promoter that contains GAL4 activation sequence. A cDNA encoded protein, fused to GAL4 activation domain, that interacts with bait gene product will reconstitute an active GAL4 protein and thereby drive expression of the lacZ gene. Colonies that express lacZ can be detected by their blue color in the presence of X-gal. The cDNA can then be purified from these strains, and used to produce and isolate the bait gene-interacting protein using techniques routinely practiced in the art. Once a pathway gene has been identified and isolated, it may be further characterized, as described below.

Characterization of Differentially Expressed and Pathway Genes

Differentially expressed and pathway genes, such as those identified via the methods discussed above, as well as genes identified by alternative means, may be further characterized by utilizing, for example, methods such as those discussed herein. Such genes will be referred to herein as “identified genes.” Analyses such as those described herein, yield information regarding the biological function of the identified genes. An assessment of the biological function of the differentially expressed genes, in addition, will allow for their designation as target and/or fingerprint genes.

Specifically, any of the differentially expressed genes whose further characterization indicates that a modulation of the gene's expression or a modulation of the gene product's activity may ameliorate any of the body weight disorders of interest will be designated “target genes,” as defined above. Such target genes and target gene products, along with those discussed below, will constitute the focus of the compound discovery strategies discussed below. Further, such target genes, target gene products and/or modulating compounds can be used as part of the body weight disorder treatment methods described below.

Any of the differentially expressed genes whose further characterization indicates that such modulations may not positively affect body weight disorders of interest, but whose expression pattern contributes to a gene expression “fingerprint” pattern correlative of, for example, a body weight disorder state will be designated a “fingerprint gene.” It should be noted that each of the target genes may also function as fingerprint genes, as may all or a portion of the pathway genes.

Pathway genes may also be characterized according to techniques such as those described herein. Those pathway genes which yield information indicating that they are differentially expressed and that modulation of the gene's expression or a modulation of the gene product's activity may ameliorate any of the body weight disorders of interest will be also be designated “target genes.” Such target genes and target gene products, along with those discussed above, will constitute the focus of the compound discovery strategies discussed below and can be used as part of treatment methods.

Characterization of one or more of the pathway genes may reveal a lack of differential expression, but evidence that modulation of the gene's activity or expression may, nonetheless, ameliorate body weight disorder symptoms. In such cases, these genes and gene products would also be considered a focus of the compound discovery strategies. In instances wherein a pathway gene's characterization indicates that modulation of gene expression or gene product activity may not positively affect body weight disorders of interest, but whose expression is differentially expressed and contributes to a gene expression fingerprint pattern correlative of, for example, a body weight disorder state, such pathway genes may additionally be designated as fingerprint genes.

A variety of techniques can be utilized to further characterize the identified genes. First, the nucleotide sequence of the identified genes, which may be obtained by utilizing standard techniques well known to those of skill in the art, may, for example, be used to reveal homologies to one or more known sequence motifs which may yield information regarding the biological function of the identified gene product.

Second, an analysis of the tissue and/or cell type distribution of the mRNA produced by the identified genes may be conducted, utilizing standard techniques well known to those of skill in the art. Such techniques may include, for example, Northern, RNase protection and RT-PCR analyses. Such analyses provide information as to, for example, whether the identified genes are expressed in tissues or cell types expected to contribute to the body weight disorders of interest. Such analyses may also provide quantitative information regarding steady state mRNA regulation, yielding data concerning which of the identified genes exhibits a high level of regulation in, preferably, tissues which may be expected to contribute to the body weight disorders of interest. Additionally, standard in situ hybridization techniques may be utilized to provide information regarding which cells within a given tissue express the identified gene. Such an analysis may provide information regarding the biological function of an identified gene relative to a given body weight disorder in instances wherein only a subset of the cells within the tissue is thought to be relevant to the body weight disorder.

Third, the sequences of the identified genes may be used, utilizing standard techniques, to place the genes onto genetic maps, e.g., mouse (Copeland and Jenkins, Trends in Genetics 7, 113-18, 1991) and human genetic maps (Cohen et al, Nature 366, 698-701, 1993). Such mapping information may yield information regarding the genes' importance to human disease by, for example, identifying genes that map within genetic regions to which known genetic body weight disorders map.

Fourth, the biological function of the identified genes may be more directly assessed by utilizing relevant in vivo and in vitro systems. In vivo systems may include, but are not limited to, animal systems that naturally exhibit body weight disorder-like symptoms, or ones which have been engineered to exhibit such symptoms. Further, such systems may include systems for the further characterization of body weight disorders, and/or appetite or body weight regulation, and may include, but are not limited to, naturally occurring and transgenic animal systems. In vitro systems may include, but are not limited to, cell-based systems comprising cell types known or suspected of contributing to the body weight disorder of interest. Such cells may be wild type cells, or may be non-wild type cells containing modifications known to, or suspected of, contributing to the body weight disorder of interest.

In further characterizing the biological function of the identified genes, the expression of these genes may be modulated within the in vivo and/or in vitro systems, i.e., either overexpressed or underexpressed in, for example, transgenic animals and/or cell lines, and its subsequent effect on the system then assayed. Alternatively, the activity of the product of the identified gene may be modulated by either increasing or decreasing the level of activity in the in vivo and/or in vitro system of interest, and its subsequent effect then assayed.

The information obtained through such characterizations may suggest relevant methods for the treatment of body weight disorders involving the gene of interest. Further, relevant methods for the control of appetite and body weight regulation involving the gene of interest may be suggested by information obtained from such characterizations. For example, treatment may include a modulation of gene expression and/or gene product activity. Characterization procedures such as those described herein may indicate where such modulation should involve an increase or a decrease in the expression or activity of the gene or gene product of interest.

Screening Methods

The invention provides assays for screening test compounds that bind to or modulate the activity of an obesity-specific polypeptide or an obesity-specific polynucleotide. A test compound preferably binds to an obesity-specific polypeptide or polynucleotide. More preferably, a test compound decreases or increases the activity of an obesity-specific polypeptide by at least about 10, preferably about 50, more preferably about 75, 90, or 100% relative to the absence of the test compound.

Test Compounds

Test compounds can be pharmacologic agents already known in the art or can be compounds previously unknown to have any pharmacological activity. Such compounds also may include, but are not limited to, other cellular proteins, peptides such as, for example, soluble peptides, including but not limited to, Ig-tailed fusion peptides, comprising extracellular portions of target gene product transmembrane receptors, and members of random peptide libraries (Lam, et al., Nature 354, 82-84, 1991; Houghten et al., Nature 354, 84-86, 1991), made of D- and/or L-configuration amino acids, phosphopeptides (including, but not limited to members of random or partially degenerate, directed phosphopeptide libraries (Songyang et al., Cell 72, 767-78, 1993), antibodies (including, but not limited to, polyclonal, monoclonal, humanized, anti-idiotypic, chimeric or single chain antibodies, and Fab, F(ab′)₂ and Fab expression library fragments, and epitope-binding fragments thereof), and small organic or inorganic molecules.

The compounds can be naturally occurring or designed in the laboratory. They can be isolated from microorganisms, animals, or plants, and can be produced recombinantly, or synthesized by chemical methods known in the art. If desired, test compounds can be obtained using any of the numerous combinatorial library methods known in the art, including but not limited to, biological libraries, spatially addressable parallel solid phase or solution phase libraries, synthetic library methods requiring deconvolution, the “one-bead one-compound” library method, and synthetic library methods using affinity chromatography selection. The biological library approach is limited to polypeptide libraries, while the other four approaches are applicable to polypeptide, non-peptide oligomer, or small molecule libraries of compounds. See Lam, Anticancer Drug Des. 12, 145, 1997.

Methods for the synthesis of molecular libraries are well known in the art (see, for example, DeWitt et al., Proc. Natl. Acad. Sci. U.S.A. 90, 6909, 1993; Erb et al. Proc. Natl. Acad. Sci. U.S.A. 91, 11422, 1994; Zuckermann et al., J. Med. Chem. 37, 2678, 1994; Cho et al., Science 261, 1303, 1993; Carell et al., Angew. Chem. Int. Ed. Engl. 33, 2059, 1994; Carell et al., Angew. Chem. Int. Ed. Engl. 33, 2061; Gallop et al., J. Med. Chem. 37, 1233, 1994). Libraries of compounds can be presented in solution (see, e.g., Houghten, BioTechniques 13, 412-421, 1992), or on beads (Lam, Nature 354, 82-84, 1991), chips (Fodor, Nature 364, 555-556, 1993), bacteria or spores (Ladner, U.S. Pat. No. 5,223,409), plasmids (Cull et al., Proc. Natl. Acad. Sci. U.S.A. 89, 1865-1869, 1992), or phage (Scott & Smith, Science 249, 386-390, 1990; Devlin, Science 249, 404-406, 1990); Cwirla et al., Proc. Natl. Acad. Sci. 97, 6378-6382, 1990; Felici, J. Mol. Biol. 222, 301-310, 1991; and Ladner, U.S. Pat. No. 5,223,409).

High Throughput Screening

Test compounds can be screened for the ability to bind to obesity-specific polypeptides or polynucleotides or to affect an obesity-specific activity or expression of an obesity-specific gene using high throughput screening. Using high throughput screening, many discrete compounds can be tested in parallel so that large numbers of test compounds can be quickly screened. The most widely established techniques utilize 96-well microtiter plates. The wells of the microtiter plates typically require assay volumes that range from 50 to 500 μl. In addition to the plates, many instruments, materials, pipettors, robotics, plate washers, and plate readers are commercially available to fit the 96-well format.

Alternatively, “free format assays,” or assays that have no physical barrier between samples, can be used. For example, an assay using pigment cells (melanocytes) in a simple homogeneous assay for combinatorial peptide libraries is described by Jayawickreme et al., Proc. Natl. Acad. Sci. U.S.A. 19, 1614-18 (1994). The cells are placed under agarose in petri dishes, then beads that carry combinatorial compounds are placed on the surface of the agarose. The combinatorial compounds are partially released the compounds from the beads. Active compounds can be visualized as dark pigment areas because, as the compounds diffuse locally into the gel matrix, the active compounds cause the cells to change colors.

Another example of a free format assay is described by Chelsky, “Strategies for Screening Combinatorial Libraries: Novel and Traditional Approaches,” reported at the First Annual Conference of The Society for Biomolecular Screening in Philadelphia, Pa. (Nov. 7-10, 1995). Chelsky placed a simple homogenous enzyme assay for carbonic anhydrase inside an agarose gel such that the enzyme in the gel would cause a color change throughout the gel. Thereafter, beads carrying combinatorial compounds via a photolinker were placed inside the gel and the compounds were partially released by UV-light. Compounds that inhibited the enzyme were observed as local zones of inhibition having less color change.

Yet another example is described by Salmon et al, Molecular Diversity 2, 57-63 (1996). In this example, combinatorial libraries were screened for compounds that had cytotoxic effects on cancer cells growing in agar.

Another high throughput screening method is described in Beutel et al., U.S. Pat. No. 5,976,813. In this method, test samples are placed in a porous matrix. One or more assay components are then placed within, on top of, or at the bottom of a matrix such as a gel, a plastic sheet, a filter, or other form of easily manipulated solid support. When samples are introduced to the porous matrix they diffuse sufficiently slowly, such that the assays can be performed without the test samples running together.

Binding Assays

For binding assays, the test compound is preferably a small molecule that binds to the obesity-specific polypeptide, such that normal biological activity is prevented. Examples of such small molecules include, but are not limited to, small peptides or peptide-like molecules.

In binding assays, either the test compound or the obesity-specific polypeptide can comprise a detectable label, such as a fluorescent, radioisotopic, chemiluminescent, or enzymatic label, such as horseradish peroxidase, alkaline phosphatase, or luciferase. Detection of a test compound that is bound to the obesity-specific polypeptide can then be accomplished, for example, by direct counting of radioemrission, by scintillation counting, or by determining conversion of an appropriate substrate to a detectable product.

Alternatively, binding of a test compound to an obesity-specific polypeptide can be determined without labeling either of the interactants. For example, a microphysiometer can be used to detect binding of a test compound with an obesity-specific polypeptide. A microphysiometer (e.g., Cytosensor™) is an analytical instrument that measures the rate at which a cell acidifies its environment using a light-addressable potentiometric sensor (LAPS). Changes in this acidification rate can be used as an indicator of the interaction between a test compound and an obesity-specific polypeptide (McConnell et al., Science 257, 1906-1912, 1992).

Determining the ability of a test compound to bind to an obesity-specific polypeptide also can be accomplished using a technology such as real-time Bimolecular Interaction Analysis (BIA) (Sjolander & Urbaniczky, Anal. Chem. 63, 2338-2345, 1991, and Szabo et al., Curr. Opin. Struct. Biol. 5, 699-705, 1995). BIA is a technology for studying biospecific interactions in real time, without labeling any of the interactants (e.g., BIAcore™). Changes in the optical phenomenon surface plasmon resonance (SPR) can be used as an indication of real-time reactions between biological molecules.

In yet another aspect of the invention, an obesity-specific polypeptide can be used as a “bait protein” in a two-hybrid assay or three-hybrid assay (see, e.g., U.S. Pat. No. 5,283,317; Zervos et al., Cell 72, 223-232, 1993; Madura et al., J. Biol. Chem. 268, 12046-12054, 1993; Bartel et al., BioTechniques 14, 920-924, 1993; Iwabuchi et al., Oncogene 8, 1693-1696, 1993; and Brent WO94/10300), to identify other proteins which bind to or interact with the an obesity-specific polypeptide and modulate its activity.

The two-hybrid system is based on the modular nature of most transcription factors, which consist of separable DNA-binding and activation domains. Briefly, the assay utilizes two different DNA constructs. For example, in one construct, polynucleotide encoding an obesity-specific polypeptide can be fused to a polynucleotide encoding the DNA binding domain of a known transcription factor (e.g., GAL-4). In the other construct a DNA sequence that encodes an unidentified protein (“prey” or “sample”) can be fused to a polynucleotide that codes for the activation domain of the known transcription factor. If the “bait” and the “prey” proteins are able to interact in vivo to form a protein-dependent complex, the DNA-binding and activation domains of the transcription factor are brought into close proximity. This proximity allows transcription of a reporter gene (e.g., LacZ), which is operably linked to a transcriptional regulatory site responsive to the transcription factor. Expression of the reporter gene can be detected, and cell colonies containing the functional transcription factor can be isolated and used to obtain the DNA sequence encoding the protein that interacts with the obesity-specific polypeptide.

It may be desirable to immobilize either the obesity-specific polypeptide (or polynucleotide) or the test compound to facilitate separation of bound from unbound forms of one or both of the interactants, as well as to accommodate automation of the assay. Thus, either the obesity-specific polypeptide (or polynucleotide) or the test compound can be bound to a solid support. Suitable solid supports include, but are not limited to, glass or plastic slides, tissue culture plates, microtiter wells, tubes, silicon chips, or particles such as beads (including, but not limited to, latex, polystyrene, or glass beads). Any method known in the art can be used to attach the polypeptide (or polynucleotide) or test compound to a solid support, including use of covalent and non-covalent linkages, passive absorption, or pairs of binding moieties attached respectively to the polypeptide (or polynucleotide) or test compound and the solid support. Test compounds are preferably bound to the solid support in an array, so that the location of individual test compounds can be tracked. Binding of a test compound to a polypeptide (or polynucleotide) can be accomplished in any vessel suitable for containing the reactants. Examples of such vessels include microtiter plates, test tubes, and microcentrifuge tubes.

In one embodiment, the obesity-specific polypeptide is a fusion protein comprising a domain that allows the obesity-specific polypeptide to be bound to a solid support. For example, glutathione-S-transferase fusion proteins can be adsorbed onto glutathione sepharose beads (Sigma Chemical, St. Louis, Mo.) or glutathione derivatized microtiter plates, which are then combined with the test compound or the test compound and the non-adsorbed obesity-specific polypeptide; the mixture is then incubated under conditions conducive to complex formation (e.g., at physiological conditions for salt and pH). Following incubation, the beads or microtiter plate wells are washed to remove any unbound components. Binding of the interactants can be determined either directly or indirectly, as described above. Alternatively, the complexes can be dissociated from the solid support before binding is determined.

Other techniques for immobilizing proteins or polynucleotides on a solid support also can be used in the screening assays of the invention. For example, either an obesity-specific polypeptide (or polynucleotide) or a test compound can be immobilized utilizing conjugation of biotin and streptavidin. Biotinylated obesity-specific polypeptides (or polynucleotides) or test compounds can be prepared from biotin-NHS(N-hydroxysuccinimide) using techniques well known in the art (e.g., biotinylation kit, Pierce Chemicals, Rockford, Ill.) and immobilized in the wells of streptavidin-coated 96 well plates (Pierce Chemical). Alternatively, antibodies which specifically bind to an obesity-specific polypeptide, polynucleotide, or a test compound, but which do not interfere with a desired binding site, such as the ATP/GTP binding site or the active site of the obesity-specific polypeptide, can be derivatized to the wells of the plate. Unbound target or protein can be trapped in the wells by antibody conjugation.

Methods for detecting such complexes, in addition to those described above for the GST-immobilized complexes, include immunodetection of complexes using antibodies which specifically bind to the obesity-specific polypeptide or test compound, enzyme-linked assays which rely on detecting an activity of the obesity-specific polypeptide, and SDS gel electrophoresis under non-reducing conditions.

Screening for test compounds which bind to an obesity-specific polypeptide or polynucleotide also can be carried out in an intact cell. Any cell that comprises an obesity-specific polypeptide or polynucleotide can be used in a cell-based assay system. An obesity-specific polynucleotide can be naturally occurring in the cell or can be introduced using techniques such as those described above. Binding of the test compound to an obesity-specific polypeptide or polynucleotide is determined as described above.

Gene Expression

In another embodiment, test compounds that increase or decrease an obesity-specific gene expression are identified. An obesity-specific polynucleotide is contacted with a test compound, and the expression of an RNA or polypeptide product of the polynucleotide is determined. The level of expression of appropriate mRNA or polypeptide in the presence of the test compound is compared to the level of expression of mRNA or polypeptide in the absence of the test compound. The test compound can then be identified as a modulator of expression based on this comparison. For example, when expression of mRNA or polypeptide is greater in the presence of the test compound than in its absence, the test compound is identified as a stimulator or enhancer of the mRNA or polypeptide expression. Alternatively, when expression of the mRNA or polypeptide is less in the presence of the test compound than in its absence, the test compound is identified as an inhibitor of the mRNA or polypeptide expression.

The level of obesity-specific mRNA or polypeptide expression in the cells can be determined by methods well known in the art for detecting mRNA or polypeptide. Either qualitative or quantitative methods can be used. The presence of polypeptide products of an obesity-specific polynucleotide can be determined, for example, using a variety of techniques known in the art, including immunochemical methods such as radioimmunoassay, Western blotting, and immunohistochemistry. Alternatively, polypeptide synthesis can be determined in vivo, in a cell culture, or in an in vitro translation system by detecting incorporation of labeled amino acids into an obesity-specific polypeptide.

Such screening can be carried out either in a cell-free assay system or in an intact cell. Any cell that expresses an obesity-specific polynucleotide can be used in a cell-based assay system. The obesity-specific polynucleotide can be naturally occurring in the cell or can be introduced using techniques such as those described above. Either a primary culture or an established cell line, such as CHO or human embryonic kidney 293 cells, can be used.

In vitro and Animal-Based Model Systems

Various in vitro and animal-based systems can act as models for body weight disorders and thermogenesis. These systems may be used in a variety of applications. For example, the animal-based model systems can be utilized to identify differentially expressed genes via one of the paradigms described above. The model systems may be used to further characterize obesity-specific genes. Such further characterization may, for example, indicate that an obesity-specific gene is a target of yet another gene.

Second, such assays may be utilized as part of screening strategies designed to identify compounds which are capable of ameliorating body weight disorder symptoms, and/or modulating thermogenesis in mammals, as described below. Amelioration of body weight disorder symptoms can itself be brought about via regulation of thermogenesis by, for example, an increase in the expression and/or activity of obesity-specific genes or gene product.

The model systems, therefore, can be used to identify drugs, pharmaceuticals, therapies, and interventions which may be effective in modulating thermogenesis, and/or in treating body weight disorders including but not limited to obesity and cachexia, via, for example, a regulation of thermogenesis. In addition, such animal models may be used to determine the LD₅₀ and the ED₅₀ in animal subjects. Such data can be used to determine the in vivo efficacy of potential body weight disorder treatments.

Animal-Based Systems

Animal-based model systems for the study of body weight disorders may include, but are not limited to, non-recombinant and engineered transgenic animals. Non-recombinant animal models for the study of body weight disorders may include, for example, genetic models. Such genetic body disorder models may include, for example, mouse models of obesity such as mice homozygous for the autosomal recessive ob, db, or tub alleles.

Non-recombinant, non-genetic animal models of body weight disorders may include, for example, rat models in which bilateral lesions exist in the ventromedial hypothalamus, leading to hyperphagia and gross obesity, or in which ventrolateral hypothalamus lesions exist, which lead to aphagia. Further, mice which, as newborns, are fed mono-sodium-glutamate (MSG) develop obesity, and may, therefore, also be utilized as animal models for body weight disorders.

Additionally, animal models for studying body weight disorders, such as, for example, animal models exhibiting body weight disorder-like symptoms, may be engineered by utilizing, for example, obesity-specific gene sequences in conjunction with techniques for producing transgenic animals that are well known to those of skill in the art. For example, obesity-specific gene sequences may be introduced into, and overexpressed in, the genome of the animal of interest, or, if endogenous obesity-specific gene sequences are present, they may, either be overexpressed or, alternatively, may be disrupted in order to underexpress or inactivate obesity-specific gene expression.

To overexpress an obesity-specific gene sequence, the coding portion of the obesity-specific gene sequence may be ligated to a regulatory sequence that is capable of driving gene expression in the animal and cell type of interest. Such regulatory regions will be well known to those of skill in the art, and may be utilized in the absence of undue experimentation. For example, aP2 promoter sequences can be used to drive adipose tissue-specific expression (Kopecky et al., J. Clin. Invest. 96, 2914-23, 1995). Recombinant obesity-specific gene sequences, therefore, can be overexpressed in adipose tissue, for example, via aP2 promoter sequences to which they have been ligated in a manner that drives obesity-specific gene expression.

For underexpression of an endogenous obesity-specific gene sequence, such a sequence may be isolated and engineered such that when reintroduced into the genome of the animal of interest, the endogenous obesity-specific gene alleles will be inactivated. Preferably, the engineered obesity-specific gene sequence is introduced via gene targeting such that the endogenous obesity-specific sequence is disrupted upon integration of the engineered obesity-specific gene sequence into the animal's genome.

Animals of any species, including, but not limited to, mice, rats, rabbits, guinea pigs, pigs, micro-pigs, goats, and non-human primates, e.g., baboons, squirrels, monkeys, and chimpanzees may be used to generate body weight disorder animal models.

Any technique known in the art may be used to introduce an obesity-specific transgene into animals to produce the founder lines of transgenic animals. Such techniques include, but are not limited to pronuclear microinjection (Hoppe & Wagner, U.S. Pat. No. 4,873,191); retrovirus mediated gene transfer into germ lines (Van der Putten et al., 1985, Proc. Natl. Acad. Sci. U.S.A. 82, 6148-52, 1985); gene targeting in embryonic stem cells (Thompson et al., Cell 56, 313-21, 1989); electroporation of embryos (Lo, Mol Cell. Biol. 3, 1803-14, 1989); and sperm-mediated gene transfer (Lavitrano et al., 1989, Cell 57, 717-23, 1989); etc. For a review of such techniques, see Gordon, “Transgenic Animals,” Intl. Rev. Cytol. 115, 171-229, 1989.

The present invention provides for transgenic animals that carry the transgene in all their cells, as well as animals which carry the transgene in some, but not all their cells, i.e., mosaic animals. See, for example, techniques described by Jakobovits, 1994, Curr. Biol. 4, 761-63, 1994). The transgene may be integrated as a single transgene or in concatamers, e.g., head-to-head tandems or head-to-tail tandems. The transgene may also be selectively introduced into and activated in a particular cell type by following, for example, the teaching of Lasko et al. (Lasko et al., Proc. Natl. Acad. Sci. U.S.A. 89, 6232-36, 1992). The regulatory sequences required for such a cell-type specific activation will depend upon the particular cell type of interest, and will be apparent to those of skill in the art.

When it is desired that the obesity-specific transgene be integrated into the chromosomal site of the endogenous obesity-specific gene, gene targeting is preferred. Briefly, when such a technique is to be utilized, vectors containing some nucleotide sequences homologous to the endogenous obesity-specific gene of interest are designed for the purpose of integrating, via homologous recombination with chromosomal sequences, into and disrupting the function of, the nucleotide sequence of the endogenous obesity-specific gene. The transgene may also be selectively introduced into a particular cell type, thus inactivating the endogenous gene of interest in only that cell type, by following, for example, the teaching of Gu et al., Science 265, 103-06, 1994). The regulatory sequences required for such a cell-type specific inactivation will depend upon the particular cell type of interest, and will be apparent to those of skill in the art.

Once transgenic animals have been generated, the expression of the recombinant obesity-specific gene and protein may be assayed utilizing standard techniques. Initial screening may be accomplished by Southern blot analysis or PCR techniques to analyze animal tissues to assay whether integration of the transgene has taken place. The level of mRNA expression of the transgene in the tissues of the transgenic animals may also be assessed using techniques which include but are not limited to Northern blot analysis of tissue samples obtained from the animal, in situ hybridization analysis, and RT-PCR. Samples of obesity-specific gene-expressing tissue, may also be evaluated immunocytochemically using antibodies specific for the obesity-specific transgene gene product of interest.

The obesity-specific gene transgenic animals that express obesity-specific gene mRNA or obesity-specific gene transgene peptide (detected immunocytochemically, using antibodies directed against obesity-specific gene product epitopes) at easily detectable levels should then be further evaluated to identify those animals which display characteristic body weight disorder-like symptoms. Such symptoms may include, for example, obesity, anorexia, and an abnormal food intake. Additionally, specific cell types within the transgenic animals may be analyzed and assayed for cellular phenotypes characteristic of body weight disorders. Such cellular phenotypes may include, for example, abnormal adipocyte differentiation (e.g., abnormal preadipocyte/adipocyte differentiation) and metabolism and/or abnormal uncoupling of oxidative phosphorylation. Further, such cellular phenotypes may include as assessment of a particular cell type's fingerprint pattern of expression and its comparison to known fingerprint expression profiles of the particular cell type in animals exhibiting body weight disorders. Such transgenic animals serve as suitable model systems for body weight disorders.

Once obesity-specific gene transgenic founder animals are produced (i.e., those animals which express obesity-specific gene proteins in cells or tissues of interest, and which, preferably, exhibit symptoms of body weight disorders), they may be bred, inbred, outbred, or crossbred to produce colonies of the particular animal. Examples of such breeding strategies include but are not limited to, outbreeding of founder animals with more than one integration site to establish separate lines; inbreeding of separate lines to produce compound obesity-specific gene transgenics that express the obesity-specific gene transgene of interest at higher levels because of the effects of additive expression of each obesity-specific gene transgene; crossing of heterozygous transgenic animals to produce animals homozygous for a given integration site in order to both augment expression and eliminate the possible need for screening of animals by DNA analysis; crossing of separate homozygous lines to produce compound heterozygous or homozygous lines; breeding animals to different inbred genetic backgrounds so as to examine effects of modifying alleles on expression of the obesity-specific gene transgene and the development of body weight disorder-like symptoms. One such approach is to cross the obesity-specific gene transgenic founder animals with a wild type strain to produce an F1 generation that exhibits body weight disorder-like symptoms, such as obesity, anorexia, abnormal food intake and/or abnormal uncoupling of oxidative phosphorylation. The F1 generation may then be inbred in order to develop a homozygous line, if it is found that homozygous obesity-specific gene transgenic animals are viable.

Cell-Based Assays

Cells that contain and express obesity-specific gene sequences which encode obesity-specific polypeptides and, further, exhibit cellular phenotypes associated with a body weight disorder of interest, may be utilized to identify compounds that exhibit an ability to ameliorate body weight disorder symptoms. Cellular phenotypes which may indicate an ability to ameliorate body weight disorders may include, for example, inhibition of adipose cell differentiation (e.g., an inhibition of differentiation of preadipocytes into adipocytes), an inhibition of the ability of adipocytes to synthesize fat and/or abnormal uncoupling of oxidative phosphorylation.

Further, the fingerprint pattern of gene expression of cells of interest may be analyzed and compared to the normal, non-body weight disorder fingerprint pattern. Those compounds which cause cells exhibiting body weight disorder-like cellular phenotypes to produce a fingerprint pattern more closely resembling a normal fingerprint pattern for the cell of interest may be considered candidates for further testing regarding an ability to ameliorate body weight disorder symptoms.

Cells that can be utilized for such assays may, for example, include non-recombinant cell lines, such as preadipocyte cell lines such as 3T3-L1 and TA1 mouse preadipocyte cell lines, liver cell lines, such as the Hepa1-6 mouse liver cell line, and the HepG2 human liver cell line.

Further, cells that may be used for such assays may also include recombinant, transgenic cell lines. For example, the body weight disorder animal models of the invention, as discussed above, may be used to generate cell lines, containing one or more cell types involved in body weight disorders, that can be used as cell culture models for this disorder. While primary cultures derived from the body weight disorder transgenic animals of the invention may be utilized, the generation of continuous cell lines is preferred. For examples of techniques that may be used to derive a continuous cell line from the transgenic animals, see Small et al., Mol. Cell Biol. 5, 642-648, 1985.

Alternatively, cells of a cell type known to be involved in body weight disorders may be transfected with sequences capable of increasing or decreasing the amount of obesity-specific gene expression within the cell. For example, obesity-specific gene sequences may be introduced into, and overexpressed in, the genome of the cell of interest, or, if endogenous obesity-specific gene sequences are present, they may either be overexpressed or, alternatively, be disrupted to underexpress or inactivate obesity-specific gene expression.

To overexpress a obesity-specific gene sequence, the coding portion of the obesity-specific gene sequence may be ligated to a regulatory sequence which is capable of driving gene expression in the cell type of interest. Such regulatory regions will be well known to those of skill in the art, and may be utilized in the absence of undue experimentation. Such sequences include, but are not limited to, aP2 promoter sequences, which drive adipose tissue-specific expression (Kopecky et al., 1995). Recombinant obesity-specific gene sequences, therefore, can be overexpressed in adipose cells, for example, via aP2 promoter sequences to which they have been ligated in a manner that drives expression.

For underexpression of an endogenous obesity-specific gene sequence, such a sequence may be isolated and engineered such that when reintroduced into the genome of the cell type of interest, the endogenous obesity-specific gene alleles will be inactivated. Preferably, the engineered obesity-specific gene sequence is introduced via gene targeting such that the endogenous obesity-specific sequence is disrupted upon integration of the engineered obesity-specific gene sequence into the cell's genome. Gene targeting is discussed above.

Transfection of obesity-specific gene sequence nucleic acid may be accomplished by utilizing standard techniques. Transfected cells should be evaluated for the presence of the recombinant obesity-specific gene sequences, for expression and accumulation of obesity-specific gene mRNA, and for the presence of recombinant obesity-specific gene protein production. In instances wherein a decrease in obesity-specific gene expression is desired, standard techniques may be used to demonstrate whether a decrease in endogenous obesity-specific gene expression and/or in obesity-specific gene product production is achieved.

Cell-based systems can be utilized to study biochemical processes which affect body weight regulation and body weight disorders. For example, cell-based assays can be utilized to study, e.g., identify compounds which modulate, uncoupling of oxidative phosphorylation as is, for example, associated with thermogenesis.

For example, yeast systems and assays can be utilized as models for uncoupling of oxidative phosphorylation. Such yeast systems express obesity-specific gene sequences. Uncoupling assays can be, for example, such as those described in Murdza-Inglis et al., J. Biol. Chem. 260, 7435-38, 1994, and Murdza-Inglis et al., 1991, J. Biol. Chem. 260, 11871-75, 1991.

In addition, mammalian cells expressing obesity-specific gene sequences, including but not limited to recombinant sequences, can also be utilized as models for uncoupling of oxidative phosphorylation. Assays for oxidative phosphorylation can, for example, include dye-based assays. In vitro systems can include, for example, those that utilize purified or partially purified obesity-specific gene product in a manner whereby the obesity-specific gene product exhibits at least one of its biological properties.

In instances whereby a body weight disorder situation results from a lower overall level of obesity-specific gene expression, obesity-specific gene product, and/or obesity-specific gene product activity in a cell or tissue involved in such a body weight disorder, compounds that interact with the obesity-specific gene product may include ones which accentuate or amplify the activity of the bound obesity-specific gene protein. Such compounds would bring about an effective increase in the level of obesity-specific gene activity, thus ameliorating symptoms. In instances whereby mutations within the obesity-specific gene cause aberrant obesity-specific gene proteins to be made which have a deleterious effect that leads to a body weight disorder, compounds that bind obesity-specific gene protein may be identified that inhibit the activity of the bound obesity-specific gene protein. Assays for testing the effectiveness of compounds are discussed below. Compounds identified via assays such as those described herein may be useful, for example, in elaborating the biological function of the obesity-specific gene product, for modulating thermogenesis, modulating body weight, and ameliorating body weight disorders.

Assays for Cellular Proteins

Any method suitable for detecting protein-protein interactions may be employed for identifying novel obesity-specific polypeptide-cellular or extracellular protein interactions. These methods are outlined above for the identification of pathway genes, and may be utilized herein with respect to the identification of proteins that interact with identified obesity-specific polypeptides.

Assays for Modulation of Body Weight-Related Processes

Compounds, including but not limited to, compounds such as those identified in the assay systems described above, may be tested for the ability to modulate body weight related processes, including, for example, thermogenesis, body weight regulation and body weight disorder symptoms, which may include, for example, obesity, anorexia, and/or an abnormal level of food intake. Gene product-based, cell-based and animal model-based assays for the identification of compounds exhibiting such an ability to modulate and/or ameliorate such processes are described below.

First, cell-based systems, such as those described above, may be used to identify compounds that may act to modulate and/or ameliorate such processes, including body weight disorder symptoms. For example, such cell systems may be exposed to a compound suspected of exhibiting an ability to modulate body weight-related processes such as an ability to ameliorate body weight disorder symptoms, at a sufficient concentration and for a time sufficient to elicit such an affect on body weight-related processes the exposed cells. After exposure, the cells are examined to determine whether one or more of the body weight-related processes has been altered. For example, in the case of body weight disorder-like cellular phenotypes, the cells can be examined to determine whether they have been altered to resemble a more normal or more wild type, non-body weight disorder phenotype, or a phenotype more likely to produce a lower incidence or severity of disorder symptoms.

In addition, the expression and/or activity levels of exposed cells can be assayed. For example, cells, preferably mammalian cells, that express or are capable of expressing an obesity-specific gene of the invention can be exposed to a test compound for a time sufficient to elicit an effect on body weight-related processed within the exposed cells. The level of obesity-specific gene expression (via, e.g., detecting mRNA transcript or obesity-specific gene products) can then be determined and compared to levels obtained in such cells in the absence of test compound. A difference in levels in exposed relative to unexposed cells identifies a compound capable of modulating body weight-related processes, including, for example, thermogenesis and/or body weight disorders such as obesity and cachexia.

In addition, animal-based body weight process-related systems, such as those described above, may be used to identify compounds capable of modulating body weight-related processes, including, for example, modulating body weight, modulating thermogenesis and ameliorating body weight disorder-like symptoms, such as obesity or cachexia symptoms. Such animal models may be used as test substrates for the identification of drugs, pharmaceuticals, therapies and interventions that may be effective in treating such processes and disorders. For example, animal models may be exposed to a test compound suspected of exhibiting an ability to modulate thermogenesis, modulate body weight or ameliorate body weight disorder symptoms, at a sufficient concentration and for a time sufficient to elicit such a body weight related effect in the exposed animals. The response of the animals to the exposure may be monitored by, for example, assessing the reversal of disorders associated with body weight disorders such as obesity or cachexia, by assaying uncoupling activities via, for example, procedures such as those described above, or by measuring the level or activity of the obesity-specific gene or gene product of interest.

With regard to intervention, any treatments that reverse any aspect of body weight disorder-like symptoms should be considered as candidates for human body weight disorder therapeutic intervention. Dosages of test agents may be determined by deriving dose-response curves.

Gene expression patterns may be utilized in conjunction with either cell-based or animal-based systems to assess the ability of a compound to modulate body weight-related processes such as, for example, an ability to ameliorate body weight disorder-like symptoms. For example, the expression pattern of one or more fingerprint genes may form part of a fingerprint profile that may be then be used in such an assessment. Fingerprint profiles are described below. Fingerprint profiles may be characterized for known states, either body weight disorder or normal states, within the cell- and/or animal-based model systems. Subsequently, these known fingerprint profiles may be compared to ascertain the effect a test compound has to modify such fingerprint profiles, and to cause the profile to more closely resemble that of a more desirable fingerprint. For example, administration of a compound may cause the fingerprint profile of a body weight disorder model system to more closely resemble the control system. Administration of a compound may, alternatively, cause the fingerprint profile of a control system to begin to mimic a body weight disorder state, which may, for example, be used in further characterizing the compound of interest, or may be used in the generation of additional animal models.

Pharmaceutical Compositions

The invention also provides pharmaceutical compositions that can be administered to a patient to achieve a therapeutic effect. Pharmaceutical compositions of the invention can comprise, for example, an obesity-specific polypeptide, an obesity-specific polynucleotide, ribozymes or antisense oligonucleotides, antibodies which specifically bind to an obesity-specific polypeptide, or mimetics, agonists, antagonists, or inhibitors of an obesity-specific polypeptide activity. The compositions can be administered alone or in combination with at least one other agent, such as stabilizing compound, which can be administered in any sterile, biocompatible pharmaceutical carrier, including, but not limited to, saline, buffered saline, dextrose, and water. The compositions can be administered to a patient alone, or in combination with other agents, drugs or hormones.

In addition to the active ingredients, these pharmaceutical compositions can contain suitable pharmaceutically-acceptable carriers comprising excipients and auxiliaries which facilitate processing of the active compounds into preparations which can be used pharmaceutically. Pharmaceutical compositions of the invention can be administered by any number of routes including, but not limited to, oral, intravenous, intramuscular, intra-arterial, intramedullary, intrathecal, intraventricular, transdermal, subcutaneous, intraperitoneal, intranasal, parenteral, topical, sublingual, or rectal means. Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropylmethylcellulose, or sodium carboxymethylcellulose; gums including arabic and tragacanth; and proteins such as gelatin and collagen. If desired, disintegrating or solubilizing agents can be added, such as the cross-linked polyvinyl pyrrolidone, agar, alginic acid, or a salt thereof, such as sodium alginate.

Dragee cores can be used in conjunction with suitable coatings, such as concentrated sugar solutions, which also can contain gum arabic, talc, polyvinylpyrrolidone, carbopol gel, polyethylene glycol, and/or titanium dioxide, lacquer solutions, and suitable organic solvents or solvent mixtures. Dyestuffs or pigments can be added to the tablets or dragee coatings for product identification or to characterize the quantity of active compound, i.e., dosage.

Pharmaceutical preparations that can be used orally include push-fit capsules made of gelatin, as well as soft, sealed capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds can be dissolved or suspended in suitable liquids, such as fatty oils, liquid, or liquid polyethylene glycol with or without stabilizers.

Pharmaceutical formulations suitable for parenteral administration can be formulated in aqueous solutions, preferably in physiologically compatible buffers such as Hanks' solution, Ringer's solution, or physiologically buffered saline. Aqueous injection suspensions can contain substances that increase the viscosity of the suspension, such as sodium carboxymethyl cellulose, sorbitol, or dextran. Additionally, suspensions of the active compounds can be prepared as appropriate oily injection suspensions. Suitable lipophilic solvents or vehicles include fatty oils such as sesame oil, or synthetic fatty acid esters, such as ethyl oleate or triglycerides, or liposomes. Non-lipid polycationic amino polymers also can be used for delivery. Optionally, the suspension also can contain suitable stabilizers or agents that increase the solubility of the compounds to allow for the preparation of highly concentrated solutions. For topical or nasal administration, penetrants appropriate to the particular barrier to be permeated are used in the formulation. Such penetrants are generally known in the art.

The pharmaceutical compositions of the present invention can be manufactured in any manner that is known in the art, e.g., by means of conventional mixing, dissolving, granulating, dragee-making, levigating, emulsifying, encapsulating, entrapping, or lyophilizing processes. The pharmaceutical composition can be provided as a salt and can be formed with many acids, including but not limited to, hydrochloric, sulfiric, acetic, lactic, tartaric, malic, succinic, etc. Salts tend to be more soluble in aqueous or other protonic solvents than are the corresponding free base forms. In other cases, the preferred preparation can be a lyophilized powder which can contain any or all of the following: 1-50 mM histidine, 0.1%-2% sucrose, and 2-7% mannitol, at a pH range of 4.5 to 5.5, that is combined with buffer prior to use.

Further details on techniques for formulation and administration can be found in the latest edition of REMINGTON'S PHARMACEUTICAL SCIENCES (Maack Publishing Co., Easton, Pa.). After pharmaceutical compositions have been prepared, they can be placed in an appropriate container and labeled for treatment of an indicated condition. Such labeling would include amount, frequency, and method of administration.

Therapeutic Indications and Methods

Obesity and overweight are defined as an excess of body fat relative to lean body mass. An increase in caloric intake or a decrease in energy expenditure or both can bring about this imbalance leading to surplus energy being stored as fat. Obesity is associated with important medical morbidities and an increase in mortality. The causes of obesity are poorly understood and may be due to genetic factors, environmental factors or a combination of the two to cause a positive energy balance. In contrast, anorexia and cachexia are characterized by an imbalance in energy intake versus energy expenditure leading to a negative energy balance and weight loss. Agents that either increase energy expenditure and/or decrease energy intake, absorption or storage would be useful for treating obesity, overweight, and associated comorbidities. Agents that either increase energy intake and/or decrease energy expenditure or increase the amount of lean tissue would be useful for treating cachexia, anorexia and wasting disorders.

Obesity-specific genes, translated proteins, and agents which modulate obesity-specific genes or portions of the genes or their products are useful for treating obesity, overweight, anorexia, cachexia, wasting disorders, appetite suppression, appetite enhancement, increases or decreases in satiety, modulation of body weight, and/or other eating disorders such as bulimia. Obesity-specific genes, translated proteins, and agents which modulate obesity-specific genes or portions of the genes or their products are useful for treating obesity/overweight-associated comorbidities including hypertension, type 2 diabetes, coronary artery disease, hyperlipidernia, stroke, gallbladder disease, gout, osteoarthritis, sleep apnea and respiratory problems, some types of cancer including endometrial, breast, prostate, and colon cancer, thrombolic disease, polycystic ovarian syndrome, reduced fertility, complications of pregnancy, menstrual irregularities, hirsutism, stress incontinence, and depression.

It is possible that body weight disorders may be brought about, at least in part, by an abnormal level of an obesity-specific gene or by the presence of an obesity-specific product exhibiting an abnormal activity. As such, the reduction in the level and/or activity of such obesity-specific gene products would bring about the amelioration of body weight disorder-like symptoms. Techniques for the reduction of obesity-specific gene expression levels or obesity-specific gene product activity levels are discussed above.

Alternatively, it is possible that body weight disorders may be brought about, at least in part, by the absence or reduction of the level of obesity-specific gene expression. As such, an increase in the level of obesity-specific gene expression and/or the activity of such gene products would bring about the amelioration of body weight disorder-like symptoms. Techniques for increasing obesity-specific gene expression levels are discussed above.

This invention further pertains to the use of novel agents identified by the screening assays described above. Accordingly, it is within the scope of this invention to use a test compound identified as described herein in an appropriate animal model. For example, an agent identified as described herein (e.g., a modulating agent, an antisense nucleic acid molecule, a specific antibody, ribozyme, or an obesity-specific polypeptide binding molecule) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

A reagent that affects an obesity-specific polypeptide or gene can be administered to a human cell, either in vitro or in vivo, to reduce levels of obesity-specific polypeptides. The reagent preferably binds to an expression product of an obesity-specific gene. If the expression product is a protein, the reagent is preferably an antibody. For treatment of human cells ex vivo, an antibody can be added to a preparation of stem cells that have been removed from the body. The cells can then be replaced in the same or another human body, with or without clonal propagation, as is known in the art.

In one embodiment, the reagent is delivered using a liposome. Preferably, the liposome is stable in the animal into which it has been administered for at least about 30 minutes, more preferably for at least about 1 hour, and even more preferably for at least about 24 hours. A liposome comprises a lipid composition that is capable of targeting a Eireagent, particularly a polynucleotide, to a particular site in an animal, such as a human. Preferably, the lipid composition of the liposome is capable of targeting to a specific organ of an animal, such as the lung, liver, spleen, heart brain, lymph nodes, and skin.

A liposome useful in the present invention comprises a lipid composition that is capable of fusing with the plasma membrane of the targeted cell to deliver its contents to the cell. Preferably, the transfection efficiency of a liposome is about 0.5 μg of DNA per 16 nmole of liposome delivered to about 10⁶ cells, more preferably about 1.0 μg of DNA per 16 nmole of liposome delivered to about 10⁶ cells, and even more preferably about 2.0 μg of DNA per 16 nmol of liposome delivered to about 10⁶ cells. Preferably, a liposome is between about 100 and 500 nm, more preferably between about 150 and 450 nm, and even more preferably between about 200 and 400 nm in diameter.

Suitable liposomes for use in the present invention include those liposomes standardly used in, for example, gene delivery methods known to those of skill in the art. More preferred liposomes include liposomes having a polycationic lipid composition and/or liposomes having a cholesterol backbone conjugated to polyethylene glycol. Optionally, a liposome comprises a compound capable of targeting the liposome to a particular cell type, such as a cell-specific ligand exposed on the outer surface of the liposome.

Complexing a liposome with a reagent such as an antisense oligonucleotide or ribozyme can be achieved using methods that are standard in the art (see, for example, U.S. Pat. No. 5,705,151). Preferably, from about 0.1 μg to about 10 μg of polynucleotide is combined with about 8 nmol of liposomes, more preferably from about 0.5 μg to about 5 μg of polynucleotides are combined with about 8 nmol liposomes, and even more preferably about 1.0 μg of polynucleotides is combined with about 8 nmol liposomes.

In another embodiment, antibodies can be delivered to specific tissues in vivo using receptor-mediated targeted delivery. Receptor-mediated DNA delivery techniques are taught in, for example, Findeis et al. Trends in Biotechnol. 11, 202-05 (1993); Chiou et al., GENE THERAPEUTICS: METHODS AND APPLICATIONS OF DIRECT GENE TRANSFER (J. A. Wolff, ed.) (1994); Wu & Wu, J. Biol. Chem. 263, 621-24 (1988); Wu et al., J. Biol. Chem. 269, 542-46 (1994); Zenke et al., Proc. Natl. Acad. Sci. U.S.A. 87, 3655-59 (1990); Wu et al., J. Biol. Chem. 266, 338-42 (1991).

Determination of a Therapeutically Effective Dose

The determination of a therapeutically effective dose is well within the capability of those skilled in the art. A therapeutically effective dose refers to that amount of active ingredient that increases or decreases an obesity-specific activity relative to an obesity-specific activity that occurs in the absence of the therapeutically effective dose.

For any compound, the therapeutically effective dose can be estimated initially either in cell culture assays or in animal models, usually mice, rabbits, dogs, or pigs. The animal model also can be used to determine the appropriate concentration range and route of administration. Such information can then be used to determine useful doses and routes for administration in humans.

Therapeutic efficacy and toxicity, e.g., ED₅₀ (the dose therapeutically effective in 50% of the population) and LD₅₀ (the dose lethal to 50% of the population), can be determined by standard pharmaceutical procedures in cell cultures or experimental animals. The dose ratio of toxic to therapeutic effects is the therapeutic index, and it can be expressed as the ratio, LD₅₀/ED₅₀.

Pharmaceutical compositions that exhibit large therapeutic indices are preferred. The data obtained from cell culture assays and animal studies is used in formulating a range of dosage for human use. The dosage contained in such compositions is preferably within a range of circulating concentrations that include the ED₅₀ with little or no toxicity. The dosage varies within this range depending upon the dosage form employed, sensitivity of the patient, and the route of administration.

The exact dosage will be determined by the practitioner, in light of factors related to the subject that requires treatment. Dosage and administration are adjusted to provide sufficient levels of the active ingredient or to maintain the desired effect. Factors that can be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions can be administered every 3 to 4 days, every week, or once every two weeks depending on the half-life and clearance rate of the particular formulation.

Normal dosage amounts can vary from 0.1 to 100,000 micrograms, up to a total dose of about 1 g, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

If the reagent is a single-chain antibody, polynucleotides encoding the antibody can be constructed and introduced into a cell either ex vivo or in vivo using well-established techniques including, but not limited to, transferrin-polycation-mediated DNA transfer, transfection with naked or encapsulated nucleic acids, liposome-mediated cellular fusion, intracellular transportation of DNA-coated latex beads, protoplast fusion, viral infection, electroporation, “gene gun,” and DEAE- or calcium phosphate-mediated transfection.

Effective in vivo dosages of an antibody are in the range of about 5 μg to about 50 μg/kg, about 50 μg to about 5 mg/kg, about 100 μg to about 500 μg/kg of patient body weight, and about 200 to about 250 μg/kg of patient body weight. For administration of polynucleotides encoding single-chain antibodies, effective in vivo dosages are in the range of about 100 ng to about 200 ng, 500 ng to about 50 mg, about 1 μg to about 2 mg, about 5 μg to about 500 μg, and about 20 μg to about 100 μg of DNA.

If the expression product is mRNA, the reagent is preferably an antisense oligonucleotide or a ribozyme. Polynucleotides that express antisense oligonucleotides or ribozymes can be introduced into cells by a variety of methods, as described above.

Preferably, a reagent reduces expression of an obesity-specific gene or the activity of an obesity-specific polypeptide by at least about 10, preferably about 50, more preferably about 75, 90, or 100% relative to the absence of the reagent. The effectiveness of the mechanism chosen to decrease the level of expression of an obesity-specific gene or the activity of an obesity-specific polypeptide can be assessed using methods well known in the art, such as hybridization of nucleotide probes to an obesity-specific-specific mRNA, quantitative RT-PCR, immunologic detection of an obesity-specific polypeptide, or measurement of an obesity-specific activity.

In any of the embodiments described above, any of the pharmaceutical compositions of the invention can be administered in combination with other appropriate therapeutic agents. Selection of the appropriate agents for use in combination therapy can be made by one of ordinary skill in the art, according to conventional pharmaceutical principles. The combination of therapeutic agents can act synergistically to effect the treatment or prevention of the various disorders described above. Using this approach, one may be able to achieve therapeutic efficacy with lower dosages of each agent, thus reducing the potential for adverse side effects.

Any of the therapeutic methods described above can be applied to any subject in need of such therapy, including, for example, mammals such as dogs, cats, cows, horses, rabbits, monkeys, and most preferably, humans.

Diagnostic Methods

A variety of methods may be employed for the diagnosis of body weight disorders, predisposition to body weight disorders, for monitoring the efficacy of antibody weight disorder compounds during, for example, clinical trials and for monitoring patients undergoing clinical evaluation for the treatment of such body weight disorders. Obesity-specific genes also can be used in diagnostic assays for detecting diseases and abnormalities or susceptibility to diseases and abnormalities related to the presence of mutations in the nucleic acid sequences that encode the polypeptide. For example, differences can be determined between the cDNA or genomic sequence encoding an obesity-specific polypeptide in individuals afflicted with a disease and in normal individuals. If a mutation is observed in some or all of the afflicted individuals but not in normal individuals, then the mutation is likely to be the causative agent of the disease.

Sequence differences between a reference gene and a gene having mutations can be revealed by the direct DNA sequencing method. In addition, cloned DNA segments can be employed as probes to detect specific DNA segments. The sensitivity of this method is greatly enhanced when combined with PCR. For example, a sequencing primer can be used with a double-stranded PCR product or a single-stranded template molecule generated by a modified PCR. The sequence determination is performed by conventional procedures using radiolabeled nucleotides or by automatic sequencing procedures using fluorescent tags.

Genetic testing based on DNA sequence differences can be carried out by detection of alteration in electrophoretic mobility of DNA fragments in gels with or without denaturing agents, Small sequence deletions and insertions can be visualized, for example, by high resolution gel electrophoresis. DNA fragments of different sequences can be distinguished on denaturing formamide gradient gels in which the mobilities of different DNA fragments are retarded in the gel at different positions according to their specific melting or partial melting temperatures (see, e.g., Myers et al., Science 230, 1242, 1985). Sequence changes at specific locations can also be revealed by nuclease protection assays, such as RNase and S 1 protection or the chemical cleavage method (e.g., Cotton et al., Proc. Natl. Acad. Sci. USA 85, 4397-4401, 1985). Thus, the detection of a specific DNA sequence can be performed by methods such as hybridization, RNase protection, chemical cleavage, direct DNA sequencing or the use of restriction enzymes and Southern blotting of genomic DNA. In addition to direct methods such as gel-electrophoresis and DNA sequencing, mutations can also be detected by in situ analysis.

Altered levels of an obesity-specific polypeptide also can be detected in various tissues. Assays used to detect levels of the polypeptides in a body sample, such as blood or a tissue biopsy, derived from a host are well known to those of skill in the art and include radioimmunoassays, competitive binding assays, Western blot analysis, and ELISA assays.

All patents and patent applications cited in this disclosure are expressly incorporated herein by reference. The above disclosure generally describes the present invention. A more complete understanding can be obtained by reference to the following specific examples which are provided for purposes of illustration only and are not intended to limit the scope of the invention.

### EXAMPLE 1

### Identification of Obesity-specific Genes by PCR-based Differential Subtraction Screening

To search for obesity-related novel target genes, a systematic analysis was carried out of the mRNAs whose expression is restricted or enriched in the hypothalamus in dietary induced obese rats compared with normal diet. PCR-based differential subtraction screening was the approach used to isolate cDNA clones of differentially expressed mRNAs.

To search for obesity-related genes, a model of high-fat-diet induced obese (DIO) rat was generated. Differential subtraction libraries of obese versus lean rat hypothalamic cDNAs were generated. Two thousand five hundred clones were obtained and analyzed from subtracted libraries by differential screening. Seven hundred positive clones that potentially up-regulated in obese rat hypothalamus were identified. Twenty-one novel clones were identified further by reverse Northern analysis.

### EXAMPLE 2

### Expression of Recombinant Obesity-specific Polypeptides

The Pichia pastoris expression vector pPICZB (Invitrogen, San Diego, Calif.) is used to produce large quantities of recombinant obesity-specific polypeptides in yeast. The DNA sequence encoding the obesity-specific polypeptide comprises a nucleotide sequence selected from the group consisting of SEQ ID NOS:1-21 and the complements thereof. Before insertion into vector PPICZB, the DNA sequence is modified by well-known methods in such a way that it contains at its 5′-end an initiation codon and at its 3′-end an enterokinase cleavage site, a His6 reporter tag and a termination codon. Moreover, at both termini recognition sequences for restriction endonucleases are added and after digestion of the multiple cloning site of pPICZ B with the corresponding restriction enzymes the modified DNA sequence is ligated into pPICZB. This expression vector is designed for inducible expression in Pichia pastoris, driven by a yeast promoter. The resulting pPICZ/md-His6 vector is used to transform the yeast.

The yeast is cultivated under usual conditions in 5 liter shake flasks and the recombinantly produced protein isolated from the culture by affinity chromatography (Ni-NTA-Resin) in the presence of 8 M urea. The bound polypeptide is eluted with buffer, pH 3.5, and neutralized. Separation of the polypeptide from the His6 reporter tag is accomplished by site-specific proteolysis using enterokinase (Invitrogen, San Diego, Calif.) according to manufacturer's instructions. Purified obesity-specific polypeptide is obtained.

### EXAMPLE 3

### Identification of Test Compounds that Bind to Obesity-specific Polypeptides

Purified obesity-specific polypeptides comprising a glutathione-S-transferase protein and absorbed onto glutathione-derivatized wells of 96-well microtiter plates are contacted with test compounds from a small molecule library at pH 7.0 in a physiological buffer solution. Obesity-specific polypeptides comprise an amino acid sequence encoded by one or more polynucleotides comprising one of the nucleotide sequences shown in SEQ ID NOS:1-21 or their complements. The test compounds comprise a fluorescent tag. The samples are incubated for 5 minutes to one hour. Control samples are incubated in the absence of a test compound.

The buffer solution containing the test compounds is washed from the wells. Binding of a test compound to an obesity-specific polypeptide is detected by fluorescence measurements of the contents of the wells. A test compound that increases the fluorescence in a well by at least 15% relative to fluorescence of a well in which a test compound is not incubated is identified as a compound that binds to an obesity-specific polypeptide.

### EXAMPLE 4

### Identification of a Test Compound Which Decreases Obesity-specific Gene Expression

A test compound is administered to a culture of human cells transfected with an obesity-specific expression construct and incubated at 37° C. for 10 to 45 minutes. A culture of the same type of cells that have not been transfected is incubated for the same time without the test compound to provide a negative control.

RNA is isolated from the two cultures as described in Chirgwin et al., Biochem. 18, 5294-99, 1979). Northern blots are prepared using 20 to 30 μg total RNA and hybridized with a ³²P-labeled obesity-specific polynucleotide probe at 65° C. in Express-hyb (CLONTECH). The probe comprises at least 11 contiguous nucleotides selected from a nucleotide sequence shown in SEQ ID NOS:1-21 or its complement. A test compound that decreases the obesity-specific gene signal relative to the signal obtained in the absence of the test compound is identified as an inhibitor of obesity-specific gene expression.

### EXAMPLE 5

### Treatment of Obesity with a Reagent that Specifically Binds to an Obesity-specific Gene Product

Synthesis of antisense an obesity-specific oligonucleotides comprising at least 11 contiguous nucleotides selected from SEQ ID NOS:1-21 or their complements is performed on a Pharmacia Gene Assembler series synthesizer using the phosphoramidite procedure (Uhlmann et al., Chem. Rev. 90, 534-83, 1990). Following assembly and deprotection, oligonucleotides are ethanol-precipitated twice, dried, and suspended in phosphate-buffered saline (PBS) at the desired concentration. Purity of these oligonucleotides is tested by capillary gel electrophoreses and ion exchange HPLC. Endotoxin levels in the oligonucleotide preparation are determined using the Limulus Amebocyte Assay (Bang, Biol. Bull. (Woods Hole, Mass.) 105, 361-362, 1953).

The antisense oligonucleotides are administered directly to a patient with obesity. The severity of the patient's obesity is decreased.

### EXAMPLE 6

### Differential Display

Total cellular RNA (10-50 μg) is treated with 20 Units of DNase I (Boehringer Mannheim) in the presence of 40 Units ribonuclease inhibitor (Boehringer Mannheim). After extraction with phenol/chloroform and ethanol precipitation, the RNA is dissolved in DEPC (diethyl pyrocarbonate)-treated water.

RNA (0.4-2 μg) is reverse-transcribed using Superscript™ reverse transcriptase (GIBCO/BRL). The cDNAs are then amplified by PCR on a Perkin-Ehner 9600 thermal cycler. The reaction mixtures (20 μl) include arbitrary decanucleotides and one of twelve possible T₁₁ VN sequences, wherein V represents either dG, dC, or dA, and N represents either dG, dT, dA, or dC. Parameters for the 40 cycle PCR reaction are as follows: Hold 94° C. for 2 minutes; cycle (40 rounds) 94° C. 15 seconds, 40° C. 2 minutes, ramp to 72° C. 30 seconds, hold 70° C. 5 minutes, hold 4° C.

Radiolabeled PCR amplification products are analyzed by electrophoresis on 6% denaturing polyacrylamide gels.

### EXAMPLE 7

### Identification of Genes Differentially Expressed in Response to Short-term Appetite Control Paradigms

Forty-five 8-week-old male C57B1/6J. mice are obtained from Jackson Laboratories. The mice are randomized into three groups of 15 mice each and housed individually on normal mouse chow (West et al., Am. J. Physiol. 262, R1025-32, 1992) for one week prior to initiation of the study. Group 1 mice (control) are maintained on ad lib mouse chow up until the time of sacrifice. Group 2 mice (fasted) are fasted for 24 hours prior to sacrifice (with water continuously available). Group 3 mice (fasted-refed) are fasted for 24 hours and then offered a highly palatable meal (mouse chow mixed with peanut butter) for 1 hour prior to sacrifice. All mice are weighed immediately before the initiation of the experiment and again immediately afterward.

Mice are sacrificed by CO₂ asphyxiation. Samples of hypothalamus, liver, small intestine, pancreas, stomach, and omental adipose tissue are collected and immediately frozen. Quantitative RT-PCR is performed as follows. One to two jig of total RNA is reverse transcribed with oligo dT₁₂₋₁₈ primers and Superscrip™ RNAse H-reverse transcriptase (Gibco-BRL, Gaithersburg, Md.). Briefly, RNA is combined with 1 μg oligo dT (500 μg /ml) in a total volume of 11 μl. The mixture is heated to 70° C. for 10 minutes and chilled on ice. After a brief centrifugation, RNA is reverse transcribed for one hour. Aliquots of the first strand cDNA are stored at −20° C. until just prior to use.

Expression levels are determined by PCR amplification of serial dilutions of first strand cDNA. In this procedure, cDNA is serially diluted in water. The dilutions are then batch amplified by PCR using sequence-specific primers. All PCR reactions are amplified under identical conditions. Therefore, the amount of product generated should reflect the amount of sequence template that is initially present. Five to ten-fold dilutions of cDNA are used, and enough dilutions are used such that the amount of product subsequently produced ranges from clearly visible (by UV illumination of ethidium bromide-stained gels) to below detection levels. The method described herein can distinguish ten-fold differences in expression levels.

Primers are designed for the amplification of the sequenced amplified bands, which are chosen using the program OLIGO (National Biosciences, Plymouth, Minn.). All quantitative PCR reactions are carried out in a 9600 Perkin-Elmer PCR machine. Generally, amplification conditions are as follows: 30-40 cycles consisting of a 95° C. denaturation for 30 seconds, 72° C. extension for 1 minute, 50-60° C. annealing for 30 seconds. Following cycling, reactions are extended for 10 minutes at 72° C.

Using such short-term appetite control paradigms and differential display techniques, several gene sequences are identified. The differential expression data identifies these gene sequences as corresponding to genes that may be involved in body weight disorders and/or body weight or appetite regulation.

### EXAMPLE 8

### Identification of Genes Differentially Expressed in Response to Genetic Obesity Paradigms

Ob/ob, db/db, and lean littermate control mice are used as part of genetic obesity paradigms. The mice are weighed at the end of the study, immediately prior to sacrifice. Upon sacrifice, tissues are collected from the four groups and immediately frozen. The tissues collected are hypothalamus, liver, small intestine, pancreas, stomach, epidiymal or uterine fat pads, and skeletal muscle. RNA is collected from the tissue samples and subjected to differential display.

Using such genetic obesity paradigms and differential display techniques, several gene sequences are identified. Differential expression data identifies these gene sequences as corresponding to genes that may be involved in body weight disorders and/or body weight or appetite regulation.

### EXAMPLE 9

### Identification of Genes Differentially Expressed in Response to Set-point Paradigms

Forty-five 8-week-old male C57B1/6J. mice are obtained from Jackson Laboratories. The mice are randomized into three groups of 15 mice each and housed individually on normal mouse chow (West et al., Am. J. Physiol. 262, R1025-32, 1992) for one week prior to initiation of the study. Group 1 mice (control) are maintained on ad lib mouse chow for an additional five days in order to calculate the daily food intake. Group 2 mice (underweight) then receive a fraction of normal food intake (60-90%) to reduce and maintain their body weight at approximately 80% of control values. Group 3 mice (overweight) are given a cafeteria diet so as to bring their body weights to 125% of control.

The three groups of 15 mice each are sacrificed by CO₂ euthanasia and tissues are immediately collected. Body weights of the three groups of 15 mice are taken at the time of sacrifice. All other tissue collection, RNA isolation, differential display, sequence analysis, Northern procedures, and RT-PCR quantitative analysis performed in this example are described above.

Using the set-point paradigm described in this example and differential display techniques, several gene sequences are identified. The differential expression data identifies these gene sequences as corresponding to genes that may be involved in body weight disorders and/or body weight or appetite regulation.

1. An isolated polynucleotide encoding a polypeptide that comprises an amino acid sequence encoded by a nucleotide sequence selected from the group consisting of SEQ ID NOS:1-21 and the complements thereof.

2. The isolated polynucleotide of claim 1 comprising a nucleotide sequence selected from the group consisting of SEQ ID NOS:1-21 and the complements thereof.

3. The isolated polynucleotide of claim 1 consisting of a nucleotide sequence selected from the group consisting of SEQ ID NOS:1-21 and the complements thereof.

4. The isolated polynucleotide of claim 1 which is a cDNA molecule.

5. An expression vector comprising a polynucleotide encoding a polypeptide comprising an amino acid sequence encoded by a nucleotide sequence selected from the group consisting of SEQ ID NOS:1-21 and the complements thereof.

6. The expression vector of claim 10 wherein the polynucleotide comprises a nucleotide sequence selected from the group consisting of SEQ ID NOS:1-21 and the complements thereof.

7. A host cell comprising an expression vector that encodes a polypeptide comprising an amino acid sequence encoded by a nucleotide sequence selected from the group consisting of SEQ ID NOS:1-21 and the complements thereof.

8. The host cell of claim 7 wherein the polynucleotide comprises a nucleotide sequence selected from the group consisting of SEQ ID NOS:1-21 and the complements thereof.

9. A purified polypeptide comprising an amino acid sequence encoded by a nucleotide sequence selected from the group consisting of SEQ ID NOS:1-21 and the complements thereof.

10. The purified polypeptide of claim 9 consisting of a polypeptide encoded by a polynucleotide comprising a nucleotide sequence selected from the group consisting of SEQ ID NOS:1-21 and the complements thereof.

11. A purified polypeptide comprising a first amino acid sequence that comprises at least one conservative amino acid substitution compared with a second amino acid sequence encoded by a nucleotide sequence selected from the group consisting of SEQ ID NOS:1-21 and the complements thereof, wherein expression of the polypeptide is increased in a hypothalamus of an obese rat relative to expression of the polypeptide in a hypothalamus of a non-obese rat.

12. A fusion protein comprising a polypeptide consisting of an amino acid sequence encoded by a nucleotide sequence selected from the group consisting of SEQ ID NOS:1-21 and the complements thereof.

13. A fusion protein comprising a polypeptide comprising a first amino acid sequence that comprises at least one conservative amino acid substitution compared with a second amino acid sequence encoded by a nucleotide sequence selected from the group consisting of SEQ ID NOS:1-21 and the complements thereof, wherein expression of the polypeptide is increased in a hypothalamus of an obese rat relative to expression of the polypeptide in a hypothalamus of a non-obese rat.

14. A method of producing a polypeptide comprising an amino acid sequence encoded by a nucleotide sequence selected from the group consisting of SEQ ID NOS:1-21 and the complements thereof, comprising the steps of:
culturing a host cell comprising an expression vector that encodes the polypeptide under conditions whereby the polypeptide is expressed; and
isolating the polypeptide.

15. The method of claim 14 wherein the expression vector comprises a nucleotide sequence selected from the group consisting of SEQ ID NOS:1-21 and the complements thereof.

16. A method of detecting a coding sequence for a polypeptide comprising an amino acid sequence encoded by a nucleotide sequence selected from the group consisting of SEQ ID NOS:1-21 and the complements thereof, comprising the steps of:
hybridizing to nucleic acid material of a biological sample a polynucleotide comprising 11 contiguous nucleotides of a nucleotide sequence selected from the group consisting of SEQ ID NOS:1-21 and the complements thereof, thereby forming a hybridization complex; and
detecting the hybridization complex.

17. The method of claim 16 further comprising the step of amplifying the nucleic acid material before the step of hybridizing.

18. A kit for detecting a coding sequence for a polypeptide comprising an amino acid sequence encoded by a nucleotide sequence selected from the group consisting of SEQ ID NOS:1-21 and the complements thereof, comprising:
a polynucleotide comprising 11 contiguous nucleotides of a nucleotide sequence selected from the group consisting of SEQ ID NOS:1-21 and the complements thereof; and
instructions for the method of claim 16.

19. A method of detecting a polypeptide comprising an amino acid sequence encoded by a nucleotide sequence selected from the group consisting of SEQ ID NOS:1-21 and the complements thereof, comprising the steps of:
contacting a biological sample with a reagent that specifically binds to the polypeptide to form a reagent-polypeptide complex; and
detecting the reagent-polypeptide complex.

20. The method of claim 9 wherein the reagent is an antibody.

21. A kit for detecting a polypeptide comprising an amino acid sequence encoded by a nucleotide sequence selected from the group consisting of SEQ ID NOS:1-21 and the complements thereof, comprising:
an antibody which specifically binds to the polypeptide; and
instructions for the method of claim 19.

22. A method of screening for agents that can regulate the activity of an obesity-specific polypeptide, comprising the steps of:
contacting a test compound with a first polypeptide selected from the group consisting of: (1) a second polypeptide encoded by a nucleotide sequence selected from the group consisting of SEQ ID NOS:1-21 and the complements thereof, and (2) a third polypeptide comprising at least one conservative amino acid substitution compared with the second polypeptide, wherein expression of the third polypeptide is increased in a hypothalamus of an obese rat relative to expression of the polypeptide in a hypothalamus of a non-obese rat; and
detecting binding of the test compound to the first polypeptide, wherein a test compound which binds to the first polypeptide is identified as a potential agent for regulating activity of the obesity-specific polypeptide.

23. The method of claim 22 wherein the step of contacting is in a cell.

24. The method of claim 22 wherein the cell is in vitro.

25. The method of claim 22 wherein the step of contacting is in a cell-free system.

26. The method of claim 22 wherein the polypeptide comprises a detectable label.

27. The method of claim 22 wherein the test compound comprises a detectable label.

28. The method of claim 22 wherein the polypeptide is bound to a solid support.

29. The method of claim 22 wherein the test compound is bound to a solid support.

30. A method of screening for agents that regulate an activity of an obesity-specific polypeptide, comprising the steps of:
contacting a test compound with a product encoded by a nucleotide sequence selected from the group consisting of SEQ ID NOS:1-21 and the complements thereof, and detecting binding of the test compound to the product, wherein a test compound which binds to the product is identified as a potential agent for regulating the activity of the obesity-specific polypeptide.

31. The method of claim 30 wherein the product is a polypeptide.

32. The method of claim 30 wherein the product is RNA.

33. A method of reducing expression of an obesity-specific polypeptide, comprising the step of:
contacting a cell with a reagent that specifically binds to a product encoded by a nucleotide sequence selected from the group consisting of SEQ ID NOS:1-21 and the complements thereof, whereby expression of the obesity-specific polypeptide is reduced.

34. The method of claim 33 wherein the product is a polypeptide.

35. The method of claim 34 wherein the reagent is an antibody.

36. The method of claim 33 wherein the product is RNA.

37. The method of claim 36 wherein the reagent is an antisense oligonucleotide.

38. The method of claim 36 wherein the reagent is a ribozyme.

39. The method of claim 33 wherein the cell is in vitro.

40. The method of claim 33 wherein the cell is in vivo.

41. A pharmaceutical composition, comprising:
an antibody that specifically binds to a polypeptide encoded by a nucleotide sequence selected from the group consisting of SEQ ID NOS:1-21 and the complements thereof; and
a pharmaceutically acceptable carrier.

42. A pharmaceutical composition, comprising:
an antisense oligonucleotide that specifically binds to a nucleotide sequence selected from the group consisting of SEQ ID NOS:1-21 and the complements thereof; and
a pharmaceutically acceptable carrier.

43. A pharmaceutical composition, comprising:
an expression vector encoding a polypeptide comprising an amino acid sequence encoded by a nucleotide sequence selected from the group consisting of SEQ ID NOS:1-21 and the complements thereof; and
a pharmaceutically acceptable carrier.

44. The pharmaceutical composition of claim 43 wherein the expression vector comprises a nucleotide sequence selected from the group consisting of SEQ ID NOS:1-21 and the complements thereof.

45. An antibody that specifically binds to a polypeptide encoded by a nucleotide sequence selected from the group consisting of SEQ ID NOS:1-21 and the complements thereof.

46. The antibody of claim 45 wherein the antibody is monoclonal.

47. The antibody of claim 45 wherein the antibody is polyclonal.


SEQUENCE LISTING (21 sequences)

SEQ ID NO: 1 - DNA, 545 bp, Rat
  misc_feature (1)...(545): n = A,T,C or G
tcttaaccccaggttttctgacagtgggtctgggtatatacagcccaggctagcctggaattcacaatcctctgactttagactctcaaatgctgggacaccgcacatgcttgtaatggacttcctaccaagcaagatggtccacacgcttatgagtactgccttagtcagggttcctattcttgtgatgatacaccatgaccaaaagcaagttgggggaagaaacacgtccacatcactcttcatcattgtaggaagtcaggacaggaattcaagcagggcaggaacctgaaagcangagcttangcagaggccacggaggctcanctttgttttctaacagaacccaangccactagccccaaggattggccccacccacaatatgctaggcaagtcccctaccaatttactaattaaaaaaattgccccaaangcttgcacccaagccccatctttttggganacattttcttcatgggtggntccctcctttaaaattgactancatttggtcaaaatgatttcccnccccaagggttgaa

SEQ ID NO: 2 - DNA, 637 bp, Rat
  misc_feature (1)...(637): n = A,T,C or G
cattctgtgcccaccattgaaactttgagtctccgacaaggtggctatgatgaactcgctagtttgcactgtctcatgttccactccttgagaacagagagtttgctcctctctcgtgtctaccacaggaggggagcttaaaagatgtgtgaagtgacgaatgaatgtactcatcaggttgctgaagcaccccataattctgtctcccaacatcctgtggccttccccttctggactcagagagcaaattgttacacaggccaaaatctgtgacacttggagtggtggtcanaaataatagatgtcctcaatctgcaggcattcttagtttgttttctatccagcgatnaagaagcgtggccaaaaacagcctgggaggaaagggttactttatcttactgctgacagaccatcatgaagggaaatcacngnaggaactcaaggcaggaacctagagcaggaactgaaccagagccatggagagacgctgctcactggcttgctttcattgactgctcactacctgttnggtatacaaccccaaaacatttaccaagagtggcaccacccacaggtgtcaggatcccggtgtgggtgccaaaggaggaacagcnagcatgggaaaccctaaaaanaa

SEQ ID NO: 3 - DNA, 499 bp, Rat
tcacttcgcccttgagactctgcgcttttcagagatttcaacagattgttcactgtctctggaaatgcactgcccagcatccttcccatcttctcgtagaatgctccaacacaagccactgctgccaattttgttggcaagtaggccgcagtgtcatccttacttctgataatgtcgttacatttatccagtgtctggaaaactgtgtaagtatccccaatgctatagagggctgcgaggtttttagctaacagttttcgagtaggtggtccaggcgagctgcttattagtccagttaattgttcaacaagctttttctatttttcttttacatctgtcttgttagcagcaactaggactttgtcaagaaatcttaaccattcgaagataaacactggtctctttgcttccgtgatttgcggtgagaggctggagagtgctagagcaaaaggaatttgcgtttaatgaatggcattcagaaaaaatggtaagtagattg

SEQ ID NO: 4 - DNA, 345 bp, Rat
tgctctccagcatggcactgcccaccaagacgcacactgtggccatgccgagagctgaaagaaggagaactcaagagggaacttctgatattgggaacagttctcagagtccccaaggagtcaagaaatctccacagacagactggctgctgggaaaccaggagtgacctttccctaacctattcctaaccacaagagccttgtgcaaaagctaggatgtgactctcttgtctttcaatggccagggagaggcgttctgctctgcccacagccgtgagctctgtgaagaacatgtgtgaaaagccagagtccgctaagaagcgttaggataccaagtccatgatg

SEQ ID NO: 5 - DNA, 247 bp, Rat
  misc_feature (1)...(247): n = A,T,C or G
atccccaaccactgctcagggctgcagcagggntgtgttctgtggccagaaatggggaacggacctggcttctgaggctgggcataggaagccaaggtgcagagcgaaagagacgaggtgacaggagggcagccccatgtcttctgggaactagacacttatttaggttttgagacagaatctctctgtgtagccctggctgtcctagaactcgctctgtagaccaggctggtctcaaactcacaga

SEQ ID NO: 6 - DNA, 132 bp, Rat
  misc_feature (1)...(132): n = A,T,C or G
ttaaactgtatgtttatacaaacaaataaatanttatctttttttcttgtacatatgcttaactttcttacaaaaatataaaaatgttcttttccatcaaaaacttctgagaaaacagattgaagggtttat

SEQ ID NO: 7 - DNA, 363 bp, Rat
  misc_feature (1)...(363): n = A,T,C or G
ctgagctatcacagtcttctgaaagcagcagttttnagccctcgggtttcagcaccccagccacctcacagggctgcttggactcttctgctgctaaatacaagatagctttaaaccccaggaagcagaagaagaaaacaagtttctcgtctgtgaaaggaaagcaggaagaacaactctattccgtgccatccaaagagaagactacaaccaagataaaacaagttgagcaaaaggagcagagactggacaatacagatgtggcagagtctgcgattagtggtccaccatcatgctatgaaattgtctcttctgaaggcaaggagaaaaaggataagtctgctgtgttgccgtggataagaa

SEQ ID NO: 8 - DNA, 280 bp, Rat
  misc_feature (1)...(280): n = A,T,C or G
ttagccctgatgagtctccagtgtcagtctactctcgagacctgccgggtaatgaggatgccaaccctcaggccagcatcttccccttggagctgggctctcctctggctcccgcgggtcctagcacctgctctccagacagcgtctgctgctctcctgattcttgctccggaatatcttctccgcctggacctgacctgnactcnaactgnaacgccctgaccacctgccangacctaccttccccagncttggaggaggaggaagacagtggggaaca

SEQ ID NO: 9 - DNA, 416 bp, Rat
  misc_feature (1)...(416): n = A,T,C or G
tacgttgaaagtgtagtcatccaccacgaangacatgagcatcggnaggaacttagtgatgagctctgcctccatcttgggctncttgaagacctggctgtctattaagtcccatgcaccttggcccagtgccagcagcctaaggaggaggaggaggtcgggactgtccctgggcagggtctcctggctaaccagctcctgcaagtgcctgatggtgctcagggacagagtgttgatggcnaaagggtcacacagatctgacatccatgtctgctggacccagnacctctcaccacagtgcccttcgcagtgtcctacanagagaaccaagtccaggtcccctgtggtggtggctttttgcttccangcatctgacttgaggacacactatttacagacgctttacaatggggcng

SEQ ID NO: 10 - DNA, 409 bp, Rat
ttacactcagacctttctggagatggctgctgattctgaacacattcaagctcaacgcctccactatcatgtggctttgaaccaaacatcggttaaggcgggagtgatgcaagagactaggccttacatggaagggccaagacaggagtattccttgacctccatggctgggccctgtttcagagttcatgtatgaatcttgccatcttagtaagcactgagggggtctgtggttggctcagcaacctgtggccagtctcagtcctcatgaaagtggctaagagtattctgttgttactgagacctagagcacccacccaaattatggaagatccactagttctagagcggccgcaccgcggtggagctccagcttttgttccctttagtgagggttaattgcgccgct

SEQ ID NO: 11 - DNA, 727 bp, Rat
  misc_feature (1)...(727): n = A,T,C or G
ttattgtcaggttttccacctatttccctggatactttgatggccagtactggctctggtgggtgttcttggttttaggctttctcctgtttctcagaggatttatcaattatgcaaaagttcggaagatgccagaaactttctcaaatctccccaggaccagagttctctttatttattaaagatgtcttctggcaaagcttcctgcactgatgaagtccctcgagaagcgagagcacctgcaggaagtgaatcaccaccaggatggaggaacagtcacctgctttaaaaaataaagtactgttgaaaaagatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacnagccggaagcataaagtgtaaagcctggggtgcctaatgagtgagctaactcacattaattgcgttgcgctcactgccgctttccagtcnggaaacctgtcgtgccanctgcattaatgaatccggcaacgcgcggggagaggcggtttgcnttttgggcgctcttncgcttcttgtcattgctcnctgcgcttggncgttcggctgtggnagccggnattanctccctaaagcggtaatcggtttccacagaatnggggataaccc

SEQ ID NO: 12 - DNA, 576 bp, Rat
ttactagcccttgtttccggtttctcctgtggcttctcaacaggcttctcaggagctccgttctcttcgtttctggtgggagacactacattggaccgaagctccctcagtggctggacaagaaccggagacggctccttaggaggcttctggcacacttcagcatagcttaactttcgaggttcctgtggggccacagctataggtgtatttacggtactattgcaaggtgaagcagtgtttgcccttgatggctttgtagtagttggagaagacactggcactggcactggattgagaacatcttttggagcagcggaatcctccatttgatccactagttctagagcggccgcaccgcggtggagctccagcttttgttccctttagtgagggttaattgcgcgcttggcgtaatcatggtcatagctgtttcctgtgtgaaattgttatccgctcacaattccacacaacatacgagccggaacataaagtgtaaagcctggggtgcctaatgaagtgagctactcacattaattgcgttgcctcactgcccgctttcatcgggaaaactgc

SEQ ID NO: 13 - DNA, 404 bp, Rat
  misc_feature (1)...(404): n = A,T,C or G
agggactcaaaaattgcctcatcttcagnttctgacccagtnangacagtntngggcccaacccctattttggccttatcagnccagaaccacaacccagtagctttctctnccttaattccagacacagactcnaaagtaccccctttcactgnctccacactggcctcttccttgccccanaaccanatccactanttctanagcggccgccaccgcggnggagctccaccttttgttccctttagtgagggttaattgcgcgcttggcnnaatcatggtcatagctgattcctgtgtgaaatngctatccgctcacaatttcacacaanatacgaacccgaagcataaagctgtnaancctggggtgcctaatgagtgacctaactcacattaattgcgtt

SEQ ID NO: 14 - DNA, 331 bp, Rat
  misc_feature (1)...(331): n = A,T,C or G
ggacgtcgactatccatgaacagataccgacgtcgactatccatgaacagataccgacgtcgactatccatgaacagataccgacgtcgactatccatgaacagataccgacgtcgactatccatgaacagataccgacgtcgactatccatgaacagataccgacgtcgactntccatgaacagatccgacgtcgactatccatgaacagatctgggtgcctggctggcggaggagacctnatccctgcantaaggaggtgtgccaactgctgtccttccttgttgatatgccnaggacgctctacnaggaggctgaggaggntagtgat

SEQ ID NO: 15 - DNA, 200 bp, Rat
  misc_feature (1)...(200): n = A,T,C or G
atgtaagtgcattgcctaggccagattccctccagaaaaccaatatggcctccatacgatgtaaggaccanagccacgttagggttntgcttagcantttctattgggatagcgnggctgggtgagaaagcatcgtctgcnncattcaggcacancgctggaattnccaccgacttcagcctgcggtctggactggcatc

SEQ ID NO: 16 - DNA, 661 bp, Rat
  misc_feature (1)...(661): n = A,T,C or G
agttgancgattgggtaccgggccccccctcgaggtcgacggtatcgataagcttgatatcgaattcctgcagcccgggggatcacactctccagcctctcaccgcagatagcactctccagcctctcaccgcagatagcactctccagcctctcaccgcagatagcactctccagcctctcaccgcagatagcactctccagcctctcatcgcagatagcactctccagcctctcaccgcagattagcactctccagcctctcaccgcagatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgccgctttccagtcgggaaacctgtcgtgccagctgcattaatgaatcggcaacgcgcggggagaggcggtttgcgtattgggcgctcttccgcttcctcgctcactgactcgctgcgctcggtcgttcgggctgcggcnagcggtatcagctcactcaaaagggcg

SEQ ID NO: 17 - DNA, 321 bp, Rat
  misc_feature (1)...(321): n = A,T,C or G
atggaggaattnatgaggaactctnggaagaaagacaagctgctntgtngccagcttcnagtantgaacttctnacagacttttctggcccaggaggataatactgaccagaacccatatgctctggcttctgaagatacaagtctacnnaaggcanctgaaactaaggagcantggaaggagctgaaggccnacatacatgatccactanttctagagccggccgcctccgnggtggagctccagctttatgttccctttngtgagggtnaattgcgcgcttggngtaatcatggtcatanctgtttcctgtgngaaata

SEQ ID NO: 18 - DNA, 525 bp, Rat
  misc_feature (1)...(525): n = A,T,C or G
caacttgtcatgaaatggagcctcaggccaaccagaatcttaggtaatgtagtcagtgcagaagtcaagtggggaggccagcaagctgaggagccagctacactgggacattaaggagtgtgagccggctacactgggacattagcgagtgtgagccagctacactgggacattaaggagtgtgagccagctacactgggacatcattaacgagtgtgagccagctacactgggacatcaataacgagtgtgagccagctacactgggacatcattaacnagtgtgagccggctacactgggacattaancgagtgtgagccagctacactgggacattagcgagtgtgagccagctacactgggacattagagagtgtgagccagctacactgggacattancgaagngtgagccagcttcactggacattagcgagtgngagccnctacactgggacgtagcgagtgggagcatcttcnctggacattagagaggtgagccagctcactggnacttaaggagt

SEQ ID NO: 19 - DNA, 331 bp, Rat
gatcaaccaaatgagaaccaagctaagcatttcctagcaacctcgcccccccccaactccatgtgagagccctactcctccagcattaacgggcctcctgcctgtctttgctacccagacaccggcctgctctgaaggttctctgctggaactctctgggaactggagggcatgctgaggaaggctctatagcttcctgtcttgtgtctggaagaggaaccttctcttcccaggtcagctgcaagacagaccacctgctaggccagcccagaagttcagggaggccttggaccccatggctcagatatgcggaccagttaacgtgcaggga

SEQ ID NO: 20 - DNA, 132 bp, Rat
tgttcgggttcagccagtgctatctatgaactttgtctcaaatcataagaaagtgtgcatgatgaaatggcaggttggctcggcatggaaaggtgcttgtcaccaagcttgtgacctgagcttgatttccag

SEQ ID NO: 21 - DNA, 246 bp, Rat
  misc_feature (1)...(246): n = A,T,C or G
gatatgcctactgtacagctagtgaaggctaaagagacactctccttancactggaacagagctctgtgntgtcggccatgctgcctaaaagcctacacagcaccttcactgggttatacgcctagtctgacttaaaaactcaacattttggactatacaaggagcaaaacttataataaaacctctacttttagatgaaaggcaaatcatttaggtaaaggcactttaagtaactgcagctaaat